SEQUENCE LISTING (21 sequences)

SEQ ID NO: 1 - DNA, 339 bp, Homo sapien
aagatctgcatacttcctaacagaggcttggcccgcaccaaggtccccattttcctggggatccagggagggagccgctgcctggcatgtgtggagacagaagaggggccttccctacagctggaggatgtgaacattgaggaactgtacaaaggtggtgaagaggccacacgcttcaccttcttccagagcagctcaggctccgccttcaggcttgaggctgctgcctggcctggctggttcctgtgtggcccggcagagccccagcagccagtacagctcaccaaggagagtgagccctcagcccgtaccaagttttactttgaacagagctggtag

SEQ ID NO: 2 - protein, 112 aa, Homo sapiens
KICILPNRGLARTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 3 - DNA, 459 bp, Homo sapien
  CDS (1)..(456)
atgtgttccctccccatggcaagatactacataattaaatatgcagaccagaaggctctatacacaagagatggccagctgctggtgggagatcctgttgcagacaactgctgtgcagagaagatctgcacacttcctaacagaggcttggaccgcaccaaggtccccattttcctggggatccagggagggagccgctgcctggcatgtgtggagacagaagaggggccttccctacagctggaggatgtgaacattgaggaactgtacaaaggtggtgaagaggccacacgcttcaccttcttccagagcagctcaggctccgccttcaggcttgaggccgctgcctggcctggctggttcctgtgtggcccggcagagccccagcagccagtacagctcactaaggagagtgagccctcagcccgtaccaagttttactttgaacagagctggtag

SEQ ID NO: 4 - protein, 152 aa, Homo sapien [sequence omitted]

SEQ ID NO: 5 - DNA, 21 bp, Artificial
  DNA primer
agaagatctgcatacttccta

SEQ ID NO: 6 - DNA, 19 bp, Artificial
  DNA primer
tgagcaggatgagcttggt

SEQ ID NO: 7 - DNA, 27 bp, Artificial
  DNA primer
ccatcctaatacgactcactatagggc

SEQ ID NO: 8 - DNA, 18 bp, Artificial
  DNA primer
ctccagctgtagggaagg

SEQ ID NO: 9 - DNA, 23 bp, Artificial
  DNA primer
actcactatagggctcgagcggc

SEQ ID NO: 10 - DNA, 20 bp, Artificial
  DNA primer
tcttctgtctccacacatgc

SEQ ID NO: 11 - DNA, 20 bp, Artificial
  DNA primer
caaggctaaaacgcagtttc

SEQ ID NO: 12 - DNA, 20 bp, Artificial
  DNA primer
caggaaacagctatgaccat

SEQ ID NO: 13 - DNA, 22 bp, Artificial
  DNA primer
ctatttaggtgacactatagaa

SEQ ID NO: 14 - DNA, 538 bp, Mus musculus
  CDS (29)..(487)
tgaagaccagacactcccaactgcaggaatgtgctcccttcccatggcaagatactacataatcaaggatgcacatcaaaaggctttgtacacacggaatggccagctcctgctgggagaccctgattcagacaattatagtccagagaaggtctgtatccttcctaaccgaggcctagaccgctccaaggtccccatcttcctggggatgcagggaggaagttgctgcctggcgtgtgtaaagacaagagagggacctctcctgcagctggaggatgtgaacatcgaggacctatacaagggaggtgaacaaaccacccgtttcacctttttccagagaagcttgggatctgccttcaggcttgaggctgctgcctgccctggctggtttctctgtggcccagctgagccccagcagccagtgcagctcaccaaagagagtgaaccctccacccatactgaattctactttgagatgagtcggtaaggagacataaggctggggcctcgtctagtgcccccagtctgagatcttctt

SEQ ID NO: 15 - protein, 152 aa, Mus musculus
MCSLPMARYYIIKDAHQKALYTRNGQLLLGDPDSDNYSPEKVCILPNRGLDRSKVPIFLGMQGGSCCLACVKTREGPLLQLEDVNIEDLYKGGEQTTRFTFFQRSLGSAFRLEAAACPGWFLCGPAEPQQPVQLTKESEPSTHTEFYFEMSR

SEQ ID NO: 16 - DNA, 30 bp, Artificial
  DNA primer
gtctgtatccttcctaaccgaggcctagac

SEQ ID NO: 17 - DNA, 30 bp, Artificial
  DNA primer
gtatgggtggagggttcactctctttggtg

SEQ ID NO: 18 - DNA, 30 bp, Artificial
  DNA primer
tgtctttacacacgccaggcagcaacttcc

SEQ ID NO: 19 - DNA, 30 bp, Artificial
  DNA primer
gcattagaatccaggcaccaggaactacag

SEQ ID NO: 20 - DNA, 30 bp, Artificial
  DNA primer
tccttattctgctttccagagatgctgagc

SEQ ID NO: 21 - protein, 33 aa, Artificial
  Polypeptide
RMKQIEDKIEEILSKIYHIENEIARIKKLIGER

(19) United States
(12) Patent Application Publication
(10) Pub. No.: US 2003/0104518 A1
(43) Pub. Date: Jun. 5, 2003

(54) FIL-1 theta DNAs and polypeptides

(76) Inventor: John E. Sims, Seattle, WA (US)

Correspondence Address: IMMUNEX CORPORATION, LAW DEPARTMENT, 51 UNIVERSITY STREET, SEATTLE, WA, 98101

(21) Appl. No.: 09/769,878

(22) Filed: Jan. 25, 2001

Related U.S. Application Data
(60) Provisional application No. 60/178,389, filed on Jan. 27, 2000.
(60) Provisional application No. 60/195,962, filed on Apr. 11, 2000.

(51) Int. Cl. C12P 21/02; C12N 5/06; C07H 21/04; C07K 14/745

(52) U.S. Cl. 435/69.1; 435/320.1; 435/325; 536/23.5; 530/384

(57) ABSTRACT

The invention is directed to novel, purified and isolated FIL-1 theta polypeptides and fragments thereof, the nucleic acids encoding such polypeptides, processes for production of recombinant forms of such polypeptides, antibodies generated against these polypeptides, fragmented peptides derived from these polypeptides, and uses thereof.

### BACKGROUND OF THE INVENTION

1. Field of the Invention

The invention is directed to purified and isolated FIL-1 theta polypeptides and fragments thereof, the nucleic acids encoding such polypeptides, processes for production of recombinant forms of such polypeptides, antibodies generated against these polypeptides, fragmented peptides derived from these polypeptides, and uses thereof.

2. Description of Related Art

Interleukin-1 (IL-1) is a member of a large group of cytokines whose primary function is to mediate immune and inflammatory responses. Included in the group of IL-1 family members are IL-1 alpha (IL-1α), IL-1 beta (IL-1β), IL-1 receptor antagonist (IL-1ra), IL-1ra beta, FIL-1 delta, IL-1 eta (also termed FIL-1 eta), IL-1 zeta (also termed FIL-1 zeta), and IL-1 epsilon (also termed FIL-1 epsilon) and IL-18 (previously known as IGIF and sometimes IL-1 gamma). IL-1, which is secreted by macrophages, is a mixture of largely IL-1β and some IL-1α (Abbas et al., 1994). IL-1α and IL-1β are first produced as 33 kD precursors that lack a signal sequence. These precursors are further processed by proteolytic cleavage to produce secreted active forms, each about 17 kD. Additionally, the 33 kD precursor of IL-1α is also active. IL-1α and IL-1β are the products of two different genes located on chromosome 2. Although the two forms are less than 30 percent homologous to each other, they bind to the same receptors and have similar activities.

IL-1α and IL-1β bind to a common receptor composed of a ligand binding chain, the type I IL-1 receptor (IL-1RI), and a required signaling component, the IL-1R accessory protein (AcP) (Sims et al. 1988; Greenfeder et al. 1995; Cullinan et al. 1998). Type II IL-1 receptor (IL-1RII) binds and sequesters the agonist IL-1 (especially IL-1β) without inducing any signaling response of its own (McMahan et al. 1991; Sims et al. 1993; Colotta et al. 1993; Colotta et al. 1994). IL-1α and IL-1β bind to a naturally occurring soluble proteolytic fragment of IL-1RII (sIL-1RII) (Colotta et al., 1993).

IL-1ra, a biologically inactive form of IL-1, is structurally homologous to IL-1. IL-1ra is produced with a signal sequence, which allows for efficient secretion into the extracellular region (Abbas et al., 1994). Additionally, IL-1ra binds to the type I IL-1 receptor but fails to bring about the subsequent interaction with AcP. Thus, IL-1ra blocks IL-1RI and prevents the action of the agonist IL-1 (Hannum et al. 1990; Eisenberg et al. 1990).

The major source of IL-1

is activated macrophages or mononuclear phagocytes. Other cells that produce IL-1 include epithelial and endothelial cells (Abbas et al., 1994). IL-1 secretion from macrophages occurs after the macrophage encounters and ingests gram-negative bacteria. Such bacteria contain lipopolysaccharide (LPS) molecules, also known as endotoxin, in the bacterial cell wall. LPS molecules are the active components that stimulate macrophages to produce tumor necrosis factor (TNF) and IL-1. IL-1 is produced in response to LPS and TNF production. At low concentrations, LPS stimulates macrophages and activates B-cells and other host responses needed to eliminate the bacterial infection; however, at high concentrations, LPS can cause severe tissue damage, shock, and even death.

The biological functions of IL-1 include activating vascular endothelial cells and lymphocytes, local tissue destruction, and fever (Janeway et al., 1996). At low levels, IL-1 stimulates macrophages and vascular endothelial cells to produce IL-6, upregulates molecules on the surface of vascular endothelial cells to increase leukocyte adhesion, and indirectly activates inflammatory leukocytes by stimulating mononuclear phagocytes and other cells to produce certain chemokines that activate inflammatory leukocytes. Additionally, IL-1 is involved in inflammatory responses that include induction of prostaglandins, nitric oxide synthetase, and metalloproteinases. These IL-1 functions are crucial during low level microbial infections. However, if the microbial infection escalates, IL-1 acts systemically by inducing fever, stimulating mononuclear phagocytes to produce IL-1 and IL-6, increasing the production of serum proteins from hepatocytes, and activating the coagulation system. Additionally, IL-1 does not cause hemorrhagic necrosis of tumors, suppress bone marrow stem cell division, and IL-1 is lethal to humans at high concentrations.

Given the important function of IL-1, there is a need to identify additional members of the IL-1 ligand family. In addition, in view of the continuing interest in protein research and the immune system, the discovery, identification, and roles of new proteins and their inhibitors, are at the forefront of modern molecular biology and biochemistry. Despite the growing body of knowledge, there is still a need in the art to discover the identity and function of proteins involved in cellular and immune responses.

### SUMMARY OF THE INVENTION

The present invention provides isolated nucleic acids and polypeptides encoded by the nucleic acids for an IL-1 family ligand termed “FIL-1 theta”. In one embodiment the present invention includes the DNA of SEQ ID NO:1, SEQ ID NO:3, SEQ ID NO:14 (termed FIL-1 theta), and nucleic acid molecules complementary to SEQ ID NO:1, SEQ ID NO:3 and SEQ ID NO:14. Similarly, in another aspect, the invention is directed to isolated polypeptides the include amino acid sequences SEQ ID NO:2, SEQ ID NO:4 and SEQ ID NO:15 and nucleic acid molecules encoding the polypeptides of SEQ ID NO:2, SEQ ID NO:4 and SEQ ID NO:15. Nucleic acids of SEQ ID NO:1 and SEQ ID NO:3 were cloned from human cells and encode the polypeptides of SEQ ID NO:2 and SEQ ID NO:4, respectively. SEQ ID NO:14 was cloned from mouse cells and encode the polypeptide of SEQ ID NO:15.

Single-stranded and double-stranded RNA and DNA nucleic acid molecules are encompassed by the invention, as well as nucleic acid molecules that hybridize to denatured, double-stranded DNA that includes all or a portion of SEQ ID NO:1 or SEQ ID NO:3 or SEQ ID NO:14 and/or a DNA that encodes the amino acid sequences set forth in SEQ ID NO:2, SEQ ID NO:4 or SEQ ID NO:15. Also encompassed by the present invention are isolated nucleic acid molecules that are derived by in vitro mutagenesis of nucleic acid molecules of sequence SEQ ID NO:1, SEQ ID NO:3 and SEQ ID NO:15 and that are degenerate from nucleic acid molecules of SEQ ID NO:1, SEQ ID NO:3 or SEQ ID NO:15, and that are allelic variants of DNA of the invention.

The invention also encompasses vectors or expression vectors that include at least one nucleic acid of the present invention and direct the expression of the polypeptides of the present invention. Also included are host cells transformed or transfected with at least one vector of the invention. Further encompassed by this invention are recombinant host cells in which the nucleic acid is integrated into the host cell genome.

The present invention provides processes for producing polypeptides encoded by the nucleic acids of the invention. The processes include culturing a host cell of this invention under conditions promoting expression of the polypeptide. Preferably the process further includes purifying the polypeptide.

The present invention further encompasses methods of using the nucleic acids noted above to identify nucleic acids encoding proteins having activities associated with IL-1 family ligands and receptors. Thus, the FIL-1 theta nucleic acid molecules can be used to identify FIL-1 theta receptors. Moreover, these nucleic acids can be used to identify the human chromosomes with which the nucleic acids are associated. Thus, the FIL-1 nucleic acids of SEQ ID NO:1 and SEQ ID NO:3 can be used to identify human chromosome 2, to map genes on human chromosome 2, to identify genes associated with certain diseases, syndromes, or other human conditions associated with human chromosomes 2, and to study cell signal transduction and the immune system.

The invention also encompasses the use of sense or antisense oligonucleotides from the nucleic acids of SEQ ID NO:1, SEQ ID NO:3 and SEQ ID NO:14 to inhibit the expression of the respective polynucleotide encoded by the genes of the invention.

The invention also encompasses isolated polypeptides and fragments of SEQ ID NO:2, SEQ ID NO:4 and SEQ ID NO:15 and nucleic acid molecules that encode the isolated polypeptides or fragments. The invention further encompasses methods for the production of these polypeptides, including culturing a host cell under conditions promoting expression and optionally, recovering the polypeptide from the culture medium. Especially, the expression of these polypeptides in bacteria, yeast, plant, insect, and animal cells is encompassed by the invention.

In general, the polypeptides of the invention can be used to study cellular processes such as immune regulation, cell proliferation, cell death, cell migration, cell-to-cell interaction, and inflammatory responses. In addition, these polypeptides can be used to identify proteins associated with FIL-1 theta.

In addition, the invention includes assays utilizing these polypeptides to screen for potential inhibitors of activity associated with polypeptide counter-structure molecules, and methods of using these polypeptides as therapeutic agents for the treatment of diseases mediated by polypeptide counter-structure molecules. Further, methods of using these polypeptides in the design of inhibitors (e.g., engineered receptors that act as inhibitors) thereof are also an aspect of the invention.

Further encompassed by this invention is the use of the FIL-1 theta nucleic acid sequences, predicted amino acid sequences of the polypeptide or fragments thereof, or a combination of the predicted amino acid sequences of the polypeptide and fragments thereof for use in searching an electronic database to aid in the identification of sample nucleic acids and/or proteins.

Isolated polyclonal or monoclonal antibodies that bind to these polypeptides are also encompassed by the invention, in addition to the use of these antibodies to aid in purifying the polypeptides of the invention.

### DETAILED DESCRIPTION OF THE INVENTION

In one embodiment, the present invention provides a nucleic acid having the following nucleotide sequence:

Name: FIL-1 theta (human)

1 AAGATCTGCA TACTTCCTAA CAGAGGCTTG GCCCGCACCA AGGTCCCCAT

51 TTTCCTGGGG ATCCAGGGAG GGAGCCGCTG CCTGGCATGT GTGGAGACAG

101 AAGAGGGGCC TTCCCTACAG CTGGAGGATG TGAACATTGA GGAACTGTAC

151 AAAGGTGGTG AAGAGGCCAC ACGCTTCACC TTCTTCCAGA GCAGCTCAGG

201 CTCCGCCTTC AGGCTTGAGG CTGCTGCCTG GCCTGGCTGG TTCCTGTGTG

251 GCCCGGCAGA GCCCCAGCAG CCAGTACAGC TCACCAAGGA GAGTGAGCCC

301 TCAGCCCGTA CCAAGTTTTA CTTTGAACAG AGCTGGTAG

(SEQ ID NO:1)

In another embodiment, the present invention includes a polypeptide having the following amino acid sequence, as well as nucleic acid molecules that encode the polypeptide.

Name: FIL-1 theta (polypeptide)

1 KICILPNRGL ARTKVPIFLG IQGGSRCLAC VETEEGPSLQ LEDVNIEELY

51 KGGEEATRFT FFQSSSGSAF RLEAAAWPGW FLCGPAEPQQ PVQLTKESEP

101 SARTKFYFEQ SW*

(SEQ ID NO:2)

In another embodiment, the present invention provides the following nucleic acid:

Name: Full length FIL-1 Theta

1 ATGTGTTCCC TCCCCATGGC AAGATACTAC ATAATTAAAT ATGCAGACCA

51 GAAGGCTCTA TACACAAGAG ATGGCCAGCT GCTGGTGGGA GATCCTGTTG

101 CAGACAACTG CTGTGCAGAG AAGATCTGCA CACTTCCTAA CAGAGGCTTG

151 GACCGCACCA AGGTCCCCAT TTTCCTGGGG ATCCAGGGAG GGAGCCGCTG

201 CCTGGCATGT GTGGAGACAG AAGAGGGGCC TTCCCTACAG CTGGAGGATG

251 TGAACATTGA GGAACTGTAC AAAGGTGGTG AAGAGGCCAC ACGCTTCACC

301 TTCTTCCAGA GCAGCTCAGG CTCCGCCTTC AGGCTTGAGG CCGCTGCCTG

351 GCCTGGCTGG TTCCTGTGTG GCCCGGCAGA GCCCCAGCAG CCAGTACAGC

401 TCACTAAGGA GAGTGAGCCC TCAGCCCGTA CCAAGTTTTA CTTTGAACAG

451 AGCTGGTAG

(SEQ ID NO:3)

In yet another embodiment the present invention includes the following polypeptide and polynucleotides that encode the polypeptide:

Name: Full length FIL-1 Theta

1 MCSLPMARYY IIKYADQKAL YTRDGQLLVG DPVADNCCAE KICTLPNRGL

51 DRTKVPIFLG IQGGSRCLAC VETEEGPSLQ LEDVNIEELY KGGEEATRFT

101 FFQSSSGSAF RLEAAAWPGW FLCGPAEPQQ PVQLTKESEP SARTKFYFEQ

151 SW*

(SEQ ID NO:4)

Further included in the present invention are the following mouse nucleic acid and the encoded mouse polypeptide:

Name: Full Length mouse FIL-1 theta polynucleotide:

1 TGAAGACCAG ACACTCCCAA CTGCAGGAAT GTGCTCCCTT CCCATGGCAA

51 GATACTACAT AATCAAGGAT GCACATCAAA AGGCTTTGTA CACACGGAAT

101 GGCCAGCTCC TGCTGGGAGA CCCTGATTCA GACAATTATA GTCCAGAGAA

151 GGTCTGTATC CTTCCTAACC GAGGCCTAGA CCGCTCCAAG GTCCCCATCT

201 TCCTGGGGAT GCAGGGAGGA AGTTGCTGCC TGGCGTGTGT AAAGACAAGA

251 GAGGGACCTC TCCTGCAGCT GGAGGATGTG AACATCGAGG ACCTATACAA

301 GGGAGGTGAA CAAACCACCC GTTTCACCTT TTTCCAGAGA AGCTTGGGAT

351 CTGCCTTCAG GCTTGAGGCT GCTGCCTGCC CTGGCTGGTT TCTCTGTGGC

401 CCAGCTGAGC CCCAGCAGCC AGTGCAGCTC ACCAAAGAGA GTGAACCCTC

451 CACCCATACT GAATTCTACT TTGAGATGAG TCGGTAAGGA GACATAAGGC

501 TGGGGCCTCG TCTAGTGCCC CCAGTCTGAG ATCTTCTT

(SEQ ID NO:14)

The coding region is from nucleotides 29-487.

Name: Full Length mouse FIL-1 theta polypeptide:

1 MCSLPMARYY IIKDAHQKAL YTRNGQLLLG DPDSDNYSPE KVCILPNRGL

51 DRSKVPIFLG MQGGSCCLAC VKTREGPLLQ LEDVNIEDLY KGGEQTTRFT

101 FFQRSLGSAF RLEAAACPGW FLCGPAEPQQ PVQLTKESEP STHTEFYFEM

151 SR*

(SEQ ID NO:15)

The discovery of the FIL-1 theta nucleic acids of the invention enables the construction of expression vectors that include nucleic acids that encode the respective polypeptides and host cells transfected or transformed with the expression vectors. The invention also enables the isolation and purification of biologically active FIL-1 theta polypeptides and fragments thereof. In yet another embodiment, the nucleic acids or oligonucleotides of the invention can be used as probes to identify nucleic acid encoding proteins having associated activities. Thus, FIL-1 theta can be used to identify activities associated with IL-1 family ligands. Additionally, nucleic acids or oligonucleotides of the present invention can be used to identify human chromosome 2. Similarly, these nucleic acids and oligonucleotides can be used to map genes on human chromosome 2 and to identify genes associated with certain diseases, syndromes or other human conditions associated with human chromosome 2. Accordingly, the nucleic acids and oligonucleotides that encode the polypeptides of the present invention can be used to identify glaucoma, ectodermal dysplasia, insulin-dependent diabetes mellitus, wrinkly skin syndrome, T-cell leukemia/lymphoma, and tibial muscular dystrophy. Finally, single-stranded sense or antisense oligonucleotides of the present invention can be used to inhibit expression of polynucleotides of this invention and can be used therapeutically to inhibit or treat disease associated with FIL-1 expression.

Further, the FIL-1 theta polypeptides and soluble peptide fragments of the invention can be used to activate and/or inhibit the activation of vascular endothelial cells and lymphocytes, induce and/or inhibit the induction of local tissue destruction and fever (Janeway et al., 1996), inhibit and/or stimulate macrophages and vascular endothelial cells to produce IL-6, induce and/or inhibit the induction of prostaglandins, nitric oxide synthetase, and metalloproteinases, and upregulate and/or inhibit the upregulation of molecules on the surface of vascular endothelial cells. In addition these polypeptides and fragmented peptides can also be used to induce and/or inhibit the induction of inflammatory mediators such as transcription factors NF-κB and AP-1, MAP kinases JNK and p38, COX-2, iNOS, and all of the activities stimulated by these molecules.

The polypeptides and fragments of this invention are useful for the generation of antibodies that bind to the polypeptides and/or polypeptide fragments of this invention. Preferably the antibodies are monoclonal antibodies and/or humanized antibodies. The invention further includes uses of the antibodies that include, but are not limited to, the purification of polypeptides of the present invention and to inhibit or activate activities associated with FIL-1 theta polypeptides.

As described in Example 1, the present invention includes FIL-1 theta polymorphisms that are indicated in a number of tissue sources. One set of polymorphisms is associated with three alleles at nucleotides 130-132 of SEQ ID NO:3, corresponding to amino acid 44 of SEQ ID NO:4. In one polymorphism, nucleotides 130-132 are ACA and encode a threonine at amino acid residues 44. Second and third polymorphisms are associated with ATA and ATC, at nucleotides 130-132, both of which encode isoleucine at amino acid residue 44. Another polymorphism has been identified at amino acid 51 of SEQ ID NO:4. More particularly, when nucleotides 151-153 of SEQ ID NO:3 are GAC, the encoded amino acid at residue 51 of SEQ ID NO:4 is aspartic acid and when nucleotides 151 to 153 are-GCC, the encoded amino acid is alanine. Oligonucleotides that encompass any of the alleles associated with nucleotides 151-153 and/or nucleotides 130-132 are useful for detecting polymorphisms associated with disease. Diseases include those directly caused by an aberrant version of FIL-1 theta or disease linked to one of these polymorphic markers.

Nucleic acids of the invention relate to isolated compounds that are free from contaminating endogenous material. Nucleic acid refers to a compound in the form of a separate fragment or as a component of a larger nucleic acid construct. In one embodiment, nucleic acids of the invention have been derived from DNA or RNA isolated at least once in substantially pure form and in a quantity or concentration enabling identification, manipulation, and recovery of its component nucleotides by standard biochemical methods (such as those outlined in Sambrook et al., Molecular Cloning: A Laboratory Manual, 2nd ed., Cold Spring Harbor Laboratory, Cold Spring Harbor, N.Y., 1989). The present invention additionally includes nucleic acids having nucleotide sequences that, due to the degeneracy of the genetic code, are different but encode the same polypeptide. Nucleotides that define the nucleic acids are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

Nucleic acids of the invention include DNA in both single-stranded and double-stranded form, as well as the RNA complement thereof. DNA includes, for example, cDNA, genomic DNA, chemically synthesized DNA, DNA amplified by PCR, and combinations thereof. Genomic DNA may be isolated by conventional techniques, e.g., using the cDNA of SEQ ID NO:1, SEQ ID NO:3, SEQ ID NO:14 or suitable fragments thereof, as a probe.

The DNA of the invention include full length genes as well as nucleic acids and fragments thereof. The full length gene may include the N-terminal signal peptide.

The nucleic acids of the invention are preferentially derived from human sources, but the invention includes those derived from non-human species, as well.

Preferred polypeptides of the invention include those encoded by the nucleic acids of SEQ ID NO:1, SEQ IDNO:3 and SEQ ID NO:14. The encoded polypeptides are shown in SEQ ID NO:2, SEQ ID NO:4, and SEQ ID NO:15, respectively. SEQ ID NO:15 is a native mouse polypeptide, and SEQ ID NO:4 is a human IL-1 theta polypeptide.

SEQ ID NO:1 identifies the FIL-1 theta as a member of the IL-1 family. SEQ ID NO:3 is the full sequence of this IL-1 family member and encodes the full polypeptides shown in SEQ ID NO:4. The homology on which this is based is set forth in Table I.

TABLE I

| | | Percent identity |
|---|---|---|
| Protein | Source | to FIL-1 theta |
| IL-1 alpha | Human | 26% |
| IL-1 beta | Human | 30% |
| FIL-1 delta (Also known as | Human | 49% |
| IL-1 delta) | | |
| IL-18 (also known as IGIF or | Human | 29% |
| IL-1 gamma) | Human | |
| IL-1ra | Human | 47% |
| IL-1 ra_beta (also known as | Human | 39% |
| IL-1 H1) | | |
| FIL-1 zeta¹ | Human | 40% |
| FIL-1 eta² | Human | 37% |
| FIL-1 epsilon³ | Human | 37% |

1 Disclosed in WO 00/36108 2 Disclosed in WO 00/71720 3 Disclosed in WO 00/11174

Tissue distribution experiments demonstrated that tissues positive for human FIL-1 theta include tonsil, skin, lung, placenta, and small airway epithelium. Tissues that are negative for FIL-1 theta include small intestine, liver, grain, fetal liver, lymph node, bone marrow, kidney, pancreas, skeletal muscle, heart, prostate, ovary, thymus, spleen, leukocytes, testis, and colon.

Due to the known degeneracy of the genetic code, wherein more than one codon can encode the same amino acid, a DNA sequence can vary from that shown in SEQ ID NO:1, SEQ ID NO:3 and SEQ ID NO:14, and still encode a polypeptide having the amino acid sequence of SEQ ID NO:2, SEQ ID NO:4, and SEQ ID NO:15, respectively. Such variant DNAs can result from inadvertent mutations (e.g., occurring during PCR amplification), or can be the product of deliberate mutagenesis of a native sequence.

Accordingly, the present invention provides isolated DNAs that include: (a) DNA that includes the nucleotide sequence of SEQ ID NO:1; (b) DNA that includes the nucleotide sequence of SEQ ID NO:3; (c) DNA encoding the polypeptide of SEQ ID NO:2; (d) DNA encoding the polypeptide of SEQ ID NO:4; (e) DNA that is the complement of DNA capable of hybridizing to a DNA of (a), (b), (c) or (d) under conditions of moderate stringency and which encodes polypeptides of the invention; (f) DNA capable of hybridization to a DNA of (a) through (d) under conditions of high stringency and which encodes polypeptides of the invention, and (g) DNA which is degenerate, as a result of the genetic code, to a DNA defined in (a), (b), (c), (d), (e), or (f) and which encode polypeptides of the invention. Of course, polypeptides encoded by such DNA sequences are encompassed by the invention.

Further, the invention includes (a) DNA that includes the nucleotide sequence of SEQ ID NO:14; (b) DNA encoding the polypeptide of SEQ ID NO:15; (c) DNA that is the complement of DNA capable of hybridizing to a DNA of (a) or (b) under conditions of moderate stringency and which encodes polypeptides of the invention; (f) DNA capable of hybridization to a DNA of (a) through (d) under conditions of high stringency and which encodes polypeptides of the invention; and, (g) DNA which is degenerate, as a result of the genetic code, to a DNA defined in (a), (b), (c), (d), (e), or (f) and which encode polypeptides of the invention. Of course, polypeptides encoded by such DNA sequences are encompassed by the invention

As used herein, conditions of moderate stringency can be readily determined by those having ordinary skill in the art based on, for example, the length of the DNA. The basic conditions are set forth by Sambrook et al., Molecular Cloning: A Laboratory Manual, 2nd ed. Vol. 1, pp. 1.101-104, Cold Spring Harbor Laboratory Press, 1989, and include use of a prewashing solution for the nitrocellulose filters 5×SSC, 0.5% SDS, 1.0 mM EDTA (pH 8.0), hybridization conditions of about 50% formamide, 6×SSC at about 42° C. (or other similar hybridization solution, such as Stark's solution, in about 50% formamide at about 42° C.), and washing conditions of about 60° C., 0.5×SSC, 0.1% SDS. Conditions of high stringency can also be readily determined by the skilled artisan based on, for example, the length of the DNA. Generally, such conditions are defined as hybridization conditions as above, and with washing at approximately 68° C., 0.2×SSC, 0.1% SDS. The skilled artisan will recognize that the temperature and wash solution salt concentration can be adjusted as necessary according to factors such as the length of the probe.

Further encompassed by the present invention are DNAs encoding polypeptide fragments and polypeptides comprising inactivated N-glycosylation site(s), inactivated protease processing site(s), or conservative amino acid substitution(s), as described below.

In another embodiment, the nucleic acid molecules of the invention also comprise nucleotide sequences that are at least 80% identical to a native sequence.

Further within the scope of the present invention are embodiments in which a nucleic acid molecule comprises a sequence that is at least 90% identical, at least 95% identical, at least 98% identical, at least 99% identical, or at least 99.9% identical to a native sequence.

The percent identity may be determined by visual inspection and mathematical calculation. Alternatively, the percent identity of two nucleic acid sequences can be determined by comparing sequence information using the GAP computer program, version 6.0 described by Devereux et al., Nucl. Acids Res. 12:387, 1984, and available from the University of Wisconsin Genetics Computer Group (UWGCG). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, pp. 353-358, National Biomedical Research Foundation, 1979; (2) a penalty of 3.0 for each gap and an additional 0.10 penalty for each symbol in each gap; and (3) no penalty for end gaps. Other programs used by one skilled in the art of sequence comparison may also be used.

The invention provides isolated nucleic acids useful in the production of polypeptides and fragments, including soluble fragments of FIL-1 theta. Such polypeptides may be prepared by any of a number of conventional techniques. A DNA encoding a polypeptide of the invention, or desired fragment thereof may be subcloned into an expression vector for production of the polypeptide or fragment. Advantageously, the DNA is fused to a sequence encoding a suitable leader or signal peptide. Alternatively, the desired DNA encoding a polypeptide or fragment of the invention may be chemically synthesized using known techniques. Such encoding DNA fragments also may be produced by restriction endonuclease digestion of a full length cloned DNA, and isolated by electrophoresis on agarose gels. If necessary, oligonucleotides that reconstruct the 5′ or 3′ terminus to a desired point may be ligated to a DNA fragment generated by restriction enzyme digestion. Such oligonucleotides may additionally contain a restriction endonuclease cleavage site upstream of the desired coding sequence, and position an initiation codon (ATG) at the N-terminus of the coding sequence.

The well-known polymerase chain reaction (PCR) procedure also may be employed to isolate and amplify a DNA encoding a desired protein fragment. Oligonucleotides that define the desired termini of the DNA fragment are employed as 5′ and 3′ primers. The oligonucleotides may additionally contain recognition sites for restriction endonucleases, to facilitate insertion of the amplified DNA fragment into an expression vector. PCR techniques are described in Saiki et al., Science, 239:487, 1988; Wu et al., eds., Recombinant DNA Methodology, pp. 189-196, Academic Press, Inc., San Diego, 1989; and Innis et al., eds., PCR Protocols: A Guide to Methods and Applications, Academic Press, Inc., 1990.

The invention encompasses polypeptides and fragments thereof in various forms, including those that are naturally occurring or produced through various techniques such as procedures involving recombinant DNA technology. Such forms include, but are not limited to, derivatives, variants, and oligomers, as well as fusion proteins or fragments thereof.

The polypeptides of the invention include partial and full length proteins encoded by the nucleic acid sequences set forth above. Preferred polypeptides of FIL-1 theta, include those having the amino acid sequence of SEQ ID NO:4 and those having the amino acid sequence of SEQ ID NO:15.

In general, the use of soluble forms is advantageous for certain applications. Purification of the polypeptides from recombinant host cells is facilitated, since the soluble polypeptides are secreted from the cells. Further, soluble polypeptides are generally more suitable for intravenous administration.

The invention also provides polypeptides and fragments that retain a desired biological activity. Particular embodiments are directed to polypeptide fragments of SEQ ID NO:2, SEQ ID NO:4 and SEQ ID NO:15, that retain the ability to bind the native cognates, substrates, or counter-structure (“binding partner”). Such a fragment may be a soluble polypeptide, as described above. In another embodiment, the polypeptides and fragments advantageously include regions that are conserved in the IL-1 ligand family as described above.

Also provided herein are polypeptide fragments comprising at least 20, or at least 30, contiguous amino acids of SEQ ID NO:2 or SEQ ID NO:4 or SEQ ID NO:15. Polypeptide fragments also may be employed as immunogens, in generating antibodies.

Naturally occurring variants as well as derived variants of the polypeptides and fragments are provided herein. Variants include polypeptides that are at least 80% identical to the polypeptide of SEQ ID NO:2 and/or the polypeptide of SEQ ID NO:4 and/or the polypeptide of SEQ ID NO:15. Also contemplated are polypeptides and fragments that comprise amino acid sequences that are at least 90% identical, at least 95% identical, at least 98% identical, at least 99% identical, or at least 99.9% identical to the polypeptide of SEQ ID NO:2, SEQ ID NO:4, SEQ ID NO:15, fragments of SEQ ID NO:2, or fragments of SEQ ID NO:4 or fragments of SEQ ID NO:15. Percent identity may be determined by visual inspection and mathematical calculation. Alternatively, the percent identity of two protein sequences can be determined by comparing sequence information using the GAP computer program, based on the algorithm of Needleman and Wunsch (J. Mol. Bio. 48:443, 1970) and available from the University of Wisconsin Genetics Computer Group (UWGCG). The preferred default parameters for the GAP program include: (1) a scoring matrix, blosum62, as described by Henikoff et al., Proc. Natl. Acad. Sci. USA, 89:10915, 1992; (2) a gap weight of 12; (3) a gap length weight of 4; and (4) no penalty for end gaps. Other programs used by one skilled in the art of sequence comparison may also be used.

The variants of the invention include, for example, those that result from alternate mRNA splicing events or from proteolytic cleavage. Alternate splicing of mRNA may, for example, yield a truncated but biologically active protein, such as a naturally occurring soluble form of the protein. Variations attributable to proteolysis include, for example, differences in the N- or C-termini upon expression in different types of host cells, due to proteolytic removal of one or more terminal amino acids from the protein (generally from 1-5 terminal amino acids). Proteins in which differences in amino acid sequence are attributable to genetic polymorphism (allelic variation among individuals producing the protein) are also contemplated herein. See Example 1 for polymorphism variations for FIL-1 theta molecules.

Additional variants within the scope of the invention include polypeptides that may be modified to create derivatives thereof by forming covalent or aggregative conjugates with other chemical moieties, such as glycosyl groups, lipids, phosphate, acetyl groups and the like. Covalent derivatives may be prepared by linking the chemical moieties to functional groups on amino acid side chains or at the N-terminus or C-terminus of a polypeptide. Conjugates comprising diagnostic (detectable) or therapeutic agents attached thereto are contemplated herein, as discussed in more detail below.

Other derivatives include covalent or aggregative conjugates of the polypeptides with other proteins or polypeptides, such as by synthesis in recombinant culture as N-terminal or C-terminal fusions. Examples of fusion proteins are discussed below in connection with oligomers. Further, fusion proteins can comprise peptides added to facilitate purification and identification. Such peptides include, for example, poly-His or the antigenic identification peptides described in U.S. Pat. No. 5,011,912 and in Hopp et al., Bio/Technology, 6:1204, 1988. One such peptide is the FLAG® peptide, Asp-Tyr-Lys-Asp-Asp-Asp-Asp-Lys, which is highly antigenic and provides an epitope reversibly bound by a specific monoclonal antibody, enabling rapid assay and facile purification of expressed recombinant protein. A murine hybridoma designated 4E11 produces a monoclonal antibody that binds the FLAG® peptide in the presence of certain divalent metal cations, as described in U.S. Pat. No. 5,011,912, hereby incorporated by reference. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn.

Among the variant polypeptides provided herein are variants of native polypeptides that retain the native biological activity or the substantial equivalent thereof. One example is a variant that binds with essentially the same binding affinity as does the native form. Binding affinity can be measured by conventional procedures, e.g., as described in U.S. Pat. No. 5,512,457 and as set forth below.

Variants include polypeptides that are substantially homologous to the native form, but which have an amino acid sequence different from that of the native form because of one or more deletions, insertions or substitutions. Particular embodiments include, but are not limited to, polypeptides that comprise from one to ten deletions, insertions or substitutions of amino acid residues, when compared to a native sequence.

A given amino acid may be replaced, for example, by a residue having similar physiochemical characteristics. Examples of such conservative substitutions include substitution of one aliphatic residue for another, such as Ile, Val, Leu, or Ala for one another; substitutions of one polar residue for another, such as between Lys and Arg, Glu and Asp, or Gln and Asn; or substitutions of one aromatic residue for another, such as Phe, Trp, or Tyr for one another. Other conservative substitutions, e.g., involving substitutions of entire regions having similar hydrophobicity characteristics, are well known.

Similarly, the DNAs of the invention include variants that differ from a native DNA sequence because of one or more deletions, insertions or substitutions, but that encode a biologically active polypeptide.

The invention further includes polypeptides of the invention with or without associated native-pattern glycosylation. Polypeptides expressed in yeast or mammalian expression systems (e.g., COS-1 or COS-7 cells) can be similar to or significantly different from a native polypeptide in molecular weight and glycosylation pattern, depending upon the choice of expression system. Expression of polypeptides of the invention in bacterial expression systems, such as E. coli, provides non-glycosylated molecules. Further, a given preparation may include multiple differentially glycosylated species of the protein. Glycosyl groups can be removed through conventional methods, in particular those utilizing glycopeptidase. In general, glycosylated polypeptides of the invention can be incubated with a molar excess of glycopeptidase (Boehringer Mannheim).

Correspondingly, similar DNA constructs that encode various additions or substitutions of amino acid residues or sequences, or deletions of terminal or internal residues or sequences are encompassed by the invention. For example, N-glycosylation sites in the polypeptide extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, the Ser or Thr can by replaced with another amino acid, such as Ala. Known procedures for inactivating N-glycosylation sites in proteins include those described in U.S. Pat. No. 5,071,972 and EP 276,846, hereby incorporated by reference.

In another example of variants, sequences encoding Cys residues that are not essential for biological activity can be altered to cause the Cys residues to be deleted or replaced with other amino acids, preventing formation of incorrect intramolecular disulfide bridges upon folding or renaturation.

Other variants are prepared by modification of adjacent dibasic amino acid residues, to enhance expression in yeast systems in which KEX2 protease activity is present. EP 212,914 discloses the use of site-specific mutagenesis to inactivate KEX2 protease processing sites in a protein. KEX2 protease processing sites are inactivated by deleting, adding or substituting residues to alter Arg-Arg, Arg-Lys, and Lys-Arg pairs to eliminate the occurrence of these adjacent basic residues. Lys-Lys pairings are considerably less susceptible to KEX2 cleavage, and conversion of Arg-Lys or Lys-Arg to Lys-Lys represents a conservative and preferred approach to inactivating KEX2 sites.

Encompassed by the invention are oligomers or fusion proteins that contain FIL-1 theta polypeptides. Such oligomers may be in the form of covalently-linked or non-covalently-linked multimers, including dimers, trimers, or higher oligomers. As noted above, preferred polypeptides are soluble and thus these oligomers may comprise soluble polypeptides. In one aspect of the invention, the oligomers maintain the binding ability of the polypeptide components and provide therefor, bivalent, trivalent, etc., binding sites.

One embodiment of the invention is directed to oligomers comprising multiple polypeptides joined via covalent or non-covalent interactions between peptide moieties fused to the polypeptides. Such peptides may be peptide linkers (spacers), or peptides that have the property of promoting oligomerization. Leucine zippers and certain polypeptides derived from antibodies are among the peptides that can promote oligomerization of the polypeptides attached thereto, as described in more detail below.

As one alternative, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al., PNAS USA 88:10535, 1991; Byrn et al., Nature 344:677, 1990; and Hollenbaugh and Aruffo, “Construction of Immunoglobulin Fusion Proteins,” in Current Protocols in Immunology, Suppl. 4, pp. 10.19.1-10.19.11, 1992.

One embodiment of the present invention is directed to a dimer comprising two fusion proteins created by fusing a polypeptide of the invention to an Fc polypeptide derived from an antibody. A gene fusion encoding the polypeptide/Fc fusion protein is inserted into an appropriate expression vector. Polypeptide/Fc fusion proteins are expressed in host cells transformed with the recombinant expression vector, and allowed to assemble much like antibody molecules, whereupon interchain disulfide bonds form between the Fc moieties to yield divalent molecules.

The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included. Preferred polypeptides comprise an Fc polypeptide derived from a human IgG1 antibody.

One suitable Fc polypeptide, described in PCT application WO 93/10151, hereby incorporated by reference, is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., EMBO J. 13:3992-4001, 1994, incorporated herein by reference. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

The above-described fusion proteins comprising Fc moieties (and oligomers formed therefrom) offer the advantage of facile purification by affinity chromatography over Protein A or Protein G columns.

In other embodiments, the polypeptides of the invention may be substituted for the variable portion of an antibody heavy or light chain. If fusion proteins are made with both heavy and light chains of an antibody, it is possible to form an oligomer with as many as four polypeptide extracellular regions.

Oligomers of the present invention include fusion proteins of more than one polypeptide described herein, with or without peptide linkers (spacer peptides). Among the suitable peptide linkers are those described in U.S. Pat. Nos. 4,751,180 and 4,935,233, which are hereby incorporated by reference. A DNA sequence encoding a desired peptide linker may be inserted between, and in the same reading frame as, the DNA sequences of the invention, using any suitable conventional technique. For example, a chemically synthesized oligonucleotide encoding the linker may be ligated between the sequences. In particular embodiments, a fusion protein comprises from two to four soluble polypeptides of the invention, separated by peptide linkers.

Another method for preparing the oligomers of the invention involves use of a leucine zipper. Leucine zipper domains are peptides that promote oligomerization of the proteins in which they are found. Leucine zippers were originally identified in several DNA-binding proteins (Landschulz et al., Science 240:1759, 1988), and have since been found in a variety of different proteins. Among the known leucine zippers are naturally occurring peptides and derivatives thereof that dimerize or trimerize.

The zipper domain (also referred to herein as an oligomerizing, or oligomer-forming, domain) comprises a repetitive heptad repeat, often with four or five leucine residues interspersed with other amino acids. Examples of zipper domains are those found in the yeast transcription factor GCN4 and a heat-stable DNA-binding protein found in rat liver (C/EBP; Landschulz et al., Science, 243:1681, 1989). Two nuclear transforming proteins,fos and jun, also exhibit zipper domains, as does the gene product of the murine proto-oncogene, c-myc (Landschulz et al., Science, 240:1759, 1988). The products of the nuclear oncogenes fos and jun comprise zipper domains that preferentially form heterodimers (O'Shea et al., Science, 245:646, 1989; Turner et al., Science, 243:1689, 1989). The zipper domain is necessary for biological activity (DNA binding) in these proteins.

The fusogenic proteins of several different viruses, including paramyxovirus, coronavirus, measles virus and many retroviruses, also possess zipper domains (Buckland et al., Nature, 338:547,1989; Britton, Nature, 353:394, 1991; Delwart and Mosialos, AIDS Research and Human Retroviruses, 6:703, 1990). The zipper domains in these fusogenic viral proteins are near the transmembrane region of the proteins; it has been suggested that the zipper domains could contribute to the oligomeric structure of the fusogenic proteins. Oligomerization of fusogenic viral proteins is involved in fusion pore formation (Spruce et al, Proc. Natl. Acad. Sci. U.S.A., 88:3523, 1991). Zipper domains have also been recently reported to play a role in oligomerization of heat-shock transcription factors (Rabindran et al., Science, 259:230, 1993).

Zipper domains fold as short, parallel coiled coils. (O'Shea et al., Science, 254:539, 1991) The general architecture of the parallel coiled coil has been well characterized, with a “knobs-into-holes” packing as proposed by Crick in 1953 (Crick, Acta Crystallogr. 6:689, 1953). The dimer formed by a zipper domain is stabilized by the heptad repeat, designated (abcdefg)ₙ according to the notation of McLachlan and Stewart, J. Mol. Biol., 98:293, 1975, in which residues a and d are generally hydrophobic residues, with d being a leucine, which line up on the same face of a helix. Oppositely charged residues commonly occur at positions g and e. Thus, in a parallel coiled coil formed from two helical zipper domains, the “knobs” formed by the hydrophobic side chains of the first helix are packed into the “holes” formed between the side chains of the second helix.

The residues at position d (often leucine) contribute large hydrophobic stabilization energies, and are important for oligomer formation (Krystek et al., Int. J. Peptide Res., 38:229, 1991). Lovejoy et al., Science, 259:1288, 1993, recently reported the synthesis of a triple-stranded α-helical bundle in which the helices run up-up-down. Their studies confirmed that hydrophobic stabilization energy provides the main driving force for the formation of coiled coils from helical monomers. These studies also indicate that electrostatic interactions contribute to the stoichiometry and geometry of coiled coils. Further discussion of the structure of leucine zippers is found in Harbury et al., Science, 262:1401, 1993.

Examples of leucine zipper domains suitable for producing soluble oligomeric proteins are described in PCT application WO 94/10308, and the leucine zipper derived from lung surfactant protein D (SPD) described in Hoppe et al., FEBS Letters, 344:191, 1994, hereby incorporated by reference. The use of a modified leucine zipper that allows for stable trimerization of a heterologous protein fused thereto is described in Fanslow et al., Semin. Immunol., 6:267-278, 1994. Recombinant fusion proteins comprising a soluble polypeptide fused to a leucine zipper peptide are expressed in suitable host cells, and the soluble oligomer that forms is recovered from the culture supernatant.

Certain leucine zipper moieties preferentially form trimers. One example is a leucine zipper derived from lung surfactant protein D (SPD), as described in Hoppe et al., FEBS Letters, 344:191, 1994, and in U.S. Pat. No. 5,716,805, hereby incorporated by reference in their entirety. This lung SPD-derived leucine zipper peptide comprises the amino acid sequence Pro Asp Val Ala Ser Leu Arg Gln Gln Val Glu Ala Leu Gln Gly Gln Val Gln His Leu Gln Ala Ala Phe Ser Gln Tyr.

Another example of a leucine zipper that promotes trimerization is a peptide comprising the amino acid sequence Arg Met Lys Gln Ile Glu Asp Lys Ile Glu Glu Ile Leu Ser Lys Ile Tyr His Ile Glu Asn Glu Ile Ala Arg Ile Lys Lys Leu Ile Gly Glu Arg (SEQ ID NO:21), as described in U.S. Pat. No. 5,716,805. In one alternative embodiment, an N-terminal Asp residue is added; in another, the peptide lacks the N-terminal Arg residue.

Fragments of the foregoing zipper peptides that retain the property of promoting oligomerization may be employed as well. Examples of such fragments include, but are not limited to, peptides lacking one or two of the N-terminal or C-terminal residues presented in the foregoing amino acid sequences. Leucine zippers may be derived from naturally occurring leucine zipper peptides, e.g., via conservative substitution(s) in the native amino acid sequence, wherein the peptide's ability to promote oligomerization is retained.

Other peptides derived from naturally occurring trimeric proteins may be employed in preparing trimeric oligomers. Alternatively, synthetic peptides that promote oligomerization may be employed. In particular embodiments, leucine residues in a leucine zipper moiety are replaced by isoleucine residues. Such peptides comprising isoleucine may be referred to as isoleucine zippers, but are encompassed by the term “leucine zippers” as employed herein.

Production of Polypeptides and Polypeptide Fragments of the Invention

Expression, isolation and purification of the polypeptides and fragments of the invention may be accomplished by any suitable technique, including but not limited to the following.

An isolated nucleic acid of the invention may be operably linked to an expression control sequence such as the pDC412 or pDC314 vectors, or the pMT2 or pED expression vectors disclosed in Kaufman et al., Nucleic Acids Res. 19, 4485-4490 (1991); and Pouwels et al. Cloning Vectors: A Laboratory Manual, Elsevier, N.Y., (1985), in order to produce the polypeptide recombinantly. Many suitable expression control sequences are known in the art. General methods of expressing recombinant polypeptides are also known and are exemplified in R. Kaufman, Methods in Enzymology 185, 537-566 (1990). As used herein “operably linked” means that the nucleic acid of the invention and an expression control sequence are situated within a construct, vector, or cell in such a way that the polypeptide encoded by the nucleic acid is expressed when appropriate molecules (such as polymerases) are present. As one embodiment of the invention, at least one expression control sequence is operably linked to a nucleic acid of the invention in a recombinant host cell or progeny thereof, the nucleic acid and/or expression control sequence having been introduced into the host cell by transformation or transfection, for example, or by any other suitable method. As another embodiment of the invention, at least one expression control sequence is integrated into the genome of a recombinant host cell such that it is operably linked to a nucleic acid sequence encoding a polypeptide of the invention. In a further embodiment of the invention, at least one expression control sequence is operably linked to a nucleic acid of the invention through the action of a trans-acting factor such as a transcription factor, either in vitro or in a recombinant host cell.

In addition, a sequence encoding an appropriate signal peptide (native or heterologous) can be incorporated into expression vectors. The choice of signal peptide or leader can depend on factors such as the type of host cells in which the recombinant polypeptide is to be produced. To illustrate, examples of heterologous signal peptides that are functional in mammalian host cells include the signal sequence for interleukin-7 (IL-7) described in U.S. Pat. No. 4,965,195; the signal sequence for interleukin-2 receptor described in Cosman et al., Nature 312:768 (1984); the interleukin-4 receptor signal peptide described in EP 367,566; the type I interleukin-1 receptor signal peptide described in U.S. Pat. No. 4,968,607; and the type II interleukin-1 receptor signal peptide described in EP 460,846. A DNA sequence for a signal peptide (secretory leader) can be fused in frame to the nucleic acid sequence of the invention so that the DNA is initially transcribed, and the mRNA translated, into a fusion polypeptide comprising the signal peptide. A signal peptide that is functional in the intended host cells promotes extracellular secretion of the polypeptide. The signal peptide is cleaved from the polypeptide upon secretion of polypeptide from the cell. The skilled artisan will also recognize that the position(s) at which the signal peptide is cleaved can differ from that predicted by computer program, and can vary according to such factors as the type of host cells employed in expressing a recombinant polypeptide. A polypeptide preparation can include a mixture of polypeptide molecules having different N-terminal amino acids, resulting from cleavage of the signal peptide at more than one site.

Established methods for introducing DNA into mammalian cells are described in Kaufman, R. J., Large Scale Mammalian Cell Culture, 1990, pp. 15-69. Additional protocols using commercially available reagents, such as Lipofectamine lipid reagent (Gibco/BRL) or Lipofectamine-Plus lipid reagent, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487-511, 1990, describes several selection schemes, such as dihydrofolate reductase (DHFR) resistance. A suitable strain for DHFR selection can be CHO strain DX-B11, which is deficient in DBFR (Urlaub and Chasin, Proc. Natl. Acad. Sci. USA 77:4216-4220, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Alternatively, gene products can be obtained via homologous recombination, or “gene targeting,” techniques. Such techniques employ the introduction of exogenous transcription control elements (such as the CMV promoter or the like) in a particular predetermined site on the genome, to induce expression of the endogenous nucleic acid sequence of interest (see, for example, U.S. Pat. No. 5,272,071). The location of integration into a host chromosome or genome can be easily determined by one of skill in the art, given the known location and sequence of the gene. In a preferred embodiment, the present invention also contemplates the introduction of exogenous transcriptional control elements in conjunction with an amplifiable gene, to produce increased amounts of the gene product, again, without the need for isolation of the gene sequence itself from the host cell.

A number of types of cells may act as suitable host cells for expression of the polypeptide. Mammalian host cells include, for example, the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, BHK (ATCC CRL 10) cell lines, the CV1/EBNA cell line derived from the African green monkey kidney cell line CV1 (ATCC CCL 70) as described by McMahan et al. (EMBO J. 10: 2821, 1991), human kidney 293 cells, human epidermal A431 cells, human Colo205 cells, other transformed primate cell lines, normal diploid cells, cell strains derived from in vitro culture of primary tissue, primary explants, HL-60, U937, HaK or Jurkat cells.

Transcriptional and translational control sequences for mammalian host cell expression vectors can be excised from viral genomes. Commonly used promoter sequences and enhancer sequences are derived from polyoma virus, adenovirus 2, simian virus 40 (SV40), and human cytomegalovirus. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., Nature, 273:113, 1978; and Kaufman, Meth. in Enzymology, 1990). Smaller or larger SV40 fragments can also be used, provided the approximately 250 bp sequence extending from the Hind III site toward the Bgl I site located in the SV40 viral origin of replication site is included.

Additional control sequences shown to improve expression of heterologous genes from mammalian expression vectors include such elements as the expression augmenting sequence element (EASE) derived from CHO cells (Morris et al., Animal Cell Technology, pp. 529-534, 1997; and PCT Application WO 97/25420) and the tripartite leader (TPL) and VA gene RNAs from Adenovirus 2 (Gingeras et al., J. Biol. Chem., 257:13475-13491, 1982). The internal ribosome entry site (IRES) sequences of viral origin allows dicistronic mRNAs to be translated efficiently (Oh et al., Current Opinion in Genetics and Development, 3:295-300, 1993; and Ramesh et al., Nucleic Acids Research, 24:2697-2700, 1996). Expression of a heterologous cDNA as part of a dicistronic mRNA followed by the gene for a selectable marker (e.g. DHFR) has been shown to improve transfectability of the host and expression of the heterologous cDNA (Kaufman, Meth. in Enzymology, 1990). Exemplary expression vectors that employ dicistronic mRNAs are pTR-DC/GFP described by Mosser et al., Biotechniques, 22:150-161, 1997, and p2A5I described by Morris et al., Animal Cell Technology, pp. 529-534, 1997.

A useful high expression vector, pCAVNOT, has been described by Mosley et al., Cell, 59:335-348, 1989. Other expression vectors for use in mammalian host cells can be constructed as disclosed by Okayama et al., (Mol. Cell. Biol., 3:280, 1983. A useful system for stable high level expression of mammalian cDNAs in C127 murine mammary epithelial cells can be constructed substantially as described by Cosman et al., Mol. Immunol., 23:935, 1986. A useful high expression vector, PMLSV N1/N4, described by Cosman et al., Nature, 312:768, 1984, has been deposited as ATCC 39890. Additional useful mammalian expression vectors are described in EP-A-0367566, and in WO 91/18982, incorporated by reference herein. In yet another alternative, the vectors can be derived from retroviruses.

Another useful expression vector, pFLAG⁷, can be used. FLAG⁷ technology is centered on the fusion of a low molecular weight (1 kD), hydrophilic, FLAG⁷ marker peptide to the N-terminus of a recombinant protein expressed by pFLAG⁷ expression vectors. pDC311 is another specialized vector used for expressing proteins in CHO cells. pDC311 is characterized by a bicistronic sequence containing the gene of interest and a dihydrofolate reductase (DHFR) gene with an internal ribosome binding site for DHFR translation, an expression augmenting sequence element (EASE), the human CMV promoter, a tripartite leader sequence, and a polyadenylation site.

Alternatively, it may be possible to produce the polypeptide in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous polypeptides. Potentially suitable bacterial strains include Escherichia coli, Bacillus subtilis, Salmonella typhimurium, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain the functional polypeptide. Such covalent attachments may be accomplished using known chemical or enzymatic methods. The polypeptide may also be produced by operably linking the isolated nucleic acid of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), and Luckow and Summers, Bio/Technology 6:47 (1988). As used herein, an insect cell capable of expressing a nucleic acid of the present invention is “transformed.” Cell-free translation systems could also be employed to produce polypeptides using RNAs derived from nucleic acid constructs disclosed herein. A host cell that comprises an isolated nucleic acid of the invention, preferably operably linked to at least one expression control sequence, is a “recombinant host cell”.

Polypeptides of the invention may be prepared by culturing transformed host cells under culture conditions suitable to express the recombinant polypeptide. The resulting expressed polypeptide may then be purified from such culture (i.e., from culture medium or cell extracts) using known purification processes, such as gel filtration and ion exchange chromatography. Polypeptide purification may also include an affinity column containing agents which will bind to the polypeptide; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography. Alternatively, the polypeptide of the invention may also be expressed in a form which will facilitate purification. For example, it may be expressed as a fusion polypeptide, such as those of maltose binding polypeptide (MBP), glutathione-S-transferase (GST) or thioredoxin (TRX). Kits for expression and purification of such fusion polypeptides are commercially available from New England BioLab (Beverly, Mass.), Pharmacia (Piscataway, N.J.) and InVitrogen, respectively. The polypeptide can also be tagged with an epitope and subsequently purified by using a specific antibody directed to such epitope. One such epitope (FLAG®) is commercially available from Kodak (New Haven, Conn.). Finally, one or more reverse-phase high performance liquid chromatography (RP-HPLC) steps employing hydrophobic RP-HPLC media, e.g., silica gel having pendant methyl or other aliphatic groups, can be employed to further purify the polypeptide. Some or all of the foregoing purification steps, in various combinations, can also be employed to provide a substantially homogeneous isolated recombinant polypeptide. The polypeptide thus purified is substantially free of other mammalian polypeptides and is defined in accordance with the present invention as an “isolated polypeptide”; such isolated polypeptides of the invention include isolated antibodies that bind to FIL-1 theta polypeptides, fragments, variants, binding partners etc. The polypeptide of the invention may also be expressed as a product of transgenic animals, e.g., as a component of the milk of transgenic cows, goats, pigs, or sheep which are characterized by somatic or germ cells containing a nucleotide sequence encoding the polypeptide.

It is also possible to utilize an affinity column comprising a polypeptide-binding polypeptide of the invention, such as a monoclonal antibody generated against polypeptides of the invention, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the invention. In this aspect of the invention, polypeptide-binding polypeptides, such as the anti-polypeptide antibodies of the invention or other polypeptides that can interact with the polypeptide of the invention, can be bound to a solid phase support such as a column chromatography matrix or a similar substrate suitable for identifying, separating, or purifying cells that express polypeptides of the invention on their surface. Adherence of polypeptide-binding polypeptides of the invention to a solid phase contacting surface can be accomplished by any means, for example, magnetic microspheres can be coated with these polypeptide-binding polypeptides and held in the incubation vessel through a magnetic field. Suspensions of cell mixtures are contacted with the solid phase that has such polypeptide-binding polypeptides thereon. Cells having polypeptides of the invention on their surface bind to the fixed polypeptide-binding polypeptide and unbound cells then are washed away. This affinity-binding method is useful for purifying, screening, or separating such polypeptide-expressing cells from solution. Methods of releasing positively selected cells from the solid phase are known in the art and encompass, for example, the use of enzymes. Such enzymes are preferably non-toxic and non-injurious to the cells and are preferably directed to cleaving the cell-surface binding partner. Alternatively, mixtures of cells suspected of containing polypeptide-expressing cells of the invention first may be incubated with a biotinylated polypeptide-binding polypeptide of the invention. The resulting mixture then is passed through a column packed with avidin-coated beads, whereby the high affinity of biotin for avidin provides the binding of the polypeptide-binding cells to the beads. Use of avidin-coated beads is known in the art. See Berenson, et al. J. Cell. Biochem., 10D:239 (1986). Wash of unbound material and the release of the bound cells is performed using conventional methods.

The polypeptide may also be produced by known conventional chemical synthesis. Methods for constructing the polypeptides of the present invention by synthetic means are known to those skilled in the art. The synthetically-constructed polypeptide sequences, by virtue of sharing primary, secondary or tertiary structural and/or conformational characteristics with polypeptides may possess biological properties in common therewith, including polypeptide activity. Thus, they may be employed as biologically active or immunological substitutes for natural, purified polypeptides in screening of therapeutic compounds and in immunological processes for the development of antibodies.

The desired degree of purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Most preferably, the polypeptide of the invention is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

Useful signal peptides include the native signal peptide, or the native peptide may be replaced by a heterologous signal peptide or leader sequence. The choice of signal peptide or leader may depend on factors such as the type of host cells in which the recombinant polypeptide is to be produced. To illustrate, examples of heterologous signal peptides that are functional in mammalian host cells include the signal sequence for interleukin-7 (IL-7) described in U.S. Pat. No. 4,965,195; the signal sequence for interleukin-2 receptor described in Cosman et al., Nature, 312:768, 1984; the interleukin-4 receptor signal peptide described in EP 367,566; the type I interleukin-1 receptor signal peptide described in U.S. Pat. No. 4,968,607; and the type II interleukin-1 receptor signal peptide described in EP 460,846.

The purified polypeptides of the invention (including proteins, polypeptides, fragments, variants, oligomers, and other forms) may be tested for the ability to bind the binding partner in any suitable assay, such as a conventional binding assay. To illustrate, the polypeptide may be labeled with a detectable reagent (e.g., a radionuclide, chromophore, enzyme that catalyzes a colorimetric or fluorometric reaction, and the like). The labeled polypeptide is contacted with cells expressing the binding partner. The cells then are washed to remove unbound labeled polypeptide, and the presence of cell-bound label is determined by a suitable technique, chosen according to the nature of the label.

One example of a binding assay procedure is as follows. A recombinant expression vector containing the binding partner cDNA is constructed using methods well known in the art. CV1-EBNA-1 cells in 10 cm² dishes are transfected with the recombinant expression vector. CV-1/EBNA-1 cells (ATCC CRL 10478) constitutively express EBV nuclear antigen-1 driven from the CMV immediate-early enhancer/promoter. CV1-EBNA-1 was derived from the African Green Monkey kidney cell line CV-1 (ATCC CCL 70), as described by McMahan et al. (EMBO J. 10:2821, 1991).

The transfected cells are cultured for 24 hours, and the cells in each dish then are split into a 24-well plate. After culturing an additional 48 hours, the transfected cells (about 4×10⁴ cells/well) are washed with BM-NFDM, which is binding medium (RPMI 1640 containing 25 mg/ml bovine serum albumin, 2 mg/ml sodium azide, 20 mM Hepes pH 7.2) to which 50 mg/ml nonfat dry milk has been added. The cells then are incubated for 1 hour at 37° C. with various concentrations of, for example, a soluble polypeptide/Fc fusion protein made as set forth above. Cells then are washed and incubated with a constant saturating concentration of a ¹²⁵I-mouse anti-human IgG in binding medium, with gentle agitation for 1 hour at 37° C. After extensive washing, cells are released via trypsinization.

The mouse anti-human IgG employed above is directed against the Fc region of human IgG and can be obtained from Jackson Immunoresearch Laboratories, Inc., West Grove, Pa. The antibody is radioiodinated using the standard chloramine-T method. The antibody will bind to the Fc portion of any polypeptide/Fc protein that has bound to the cells. In all assays, non-specific binding of ¹²⁵I-antibody is assayed in the absence of the Fc fusion protein/Fc, as well as in the presence of the Fc fusion protein and a 200-fold molar excess of unlabeled mouse anti-human IgG antibody.

Cell-bound ¹²⁵I-antibody is quantified on a Packard Autogamma counter. Affinity calculations (Scatchard, Ann. N.Y. Acad. Sci., 51:660, 1949) are generated on RS/1 (BBN Software, Boston, Mass.) run on a Microvax computer.

Another type of suitable binding assay is a competitive binding assay. To illustrate, biological activity of a variant may be determined by assaying for the variant's ability to compete with the native protein for binding to the binding partner.

Competitive binding assays can be performed by conventional methodology. Reagents that may be employed in competitive binding assays include radiolabeled polypeptides of the invention and intact cells expressing the binding partner (endogenous or recombinant). For example, a radiolabeled soluble FIL-1 theta fragment can be used to compete with a soluble FIL-1 theta variant for binding to cell surface FIL-1 theta receptors. Instead of intact cells, one could substitute a soluble binding partner/Fc fusion protein bound to a solid phase through the interaction of Protein A or Protein G (on the solid phase) with the Fc moiety. Chromatography columns that contain Protein A and Protein G include those available from Pharmacia Biotech, Inc., Piscataway, N.J.

Another type of competitive binding assay utilizes radiolabeled soluble binding partner, such as a soluble FIL-1 theta receptor/Fc fusion protein, and intact cells expressing the binding partner. Qualitative results can be obtained by competitive autoradiographic plate binding assays, while Scatchard plots (Scatchard, Ann. N.Y. Acad. Sci:, 51:660, 1949) may be utilized to generate quantitative results.

Use of FIL-1 Theta Nucleic Acids or Oligonucleotides

In addition to being used to express polypeptides as described above, the nucleic acids of the invention, including DNA, RNA, mRNA, and oligonucleotides thereof can be used:

as probes to identify nucleic acid encoding proteins of the IL-1 ligand and receptor families;

to identify human chromosome 2;

to map genes on human chromosome 2;

to identify genes associated with certain diseases, syndromes, or other conditions associated with human chromosome 2;

as single-stranded sense or antisense oligonucleotides, to inhibit expression of polypeptides encoded by the FIL-1 theta gene;

to help detect defective genes in an individual; and

for gene therapy.

Nucleic acids described herein and particularly fragments of nucleic acids of this invention, oligonucleotides, find uses as probes or primers. Suitable oligonucleotide primers include those having at least about 17 contiguous nucleotides. In other embodiments, suitable oligonucleotides include at least 30, or at least 60, contiguous nucleotides of a DNA sequence of this invention. Preferably, the contiguous oligonucleotides, are fragments of SEQ ID NO:1, SEQ ID NO:3 or SEQ ID NO:14.

Because homologs of SEQ ID NO:1 or SEQ ID NO:3, from other mammalian species, e.g. SEQ ID NO:14, are contemplated herein, probes based on the human DNA of SEQ ID NO:1 or SEQ ID NO:3 may be used to screen cDNA libraries derived from other mammalian species, using conventional cross-species hybridization techniques.

Using knowledge of the genetic code in combination with the amino acid sequences set forth above, sets of degenerate oligonucleotides can be prepared. Such oligonucleotides are useful as primers, e.g., in polymerase chain reactions (PCR), whereby DNA fragments are isolated and amplified.

All or a portion of the nucleic acids of SEQ ID NO:1 or SEQ ID NO:3, including oligonucleotides, can be used by those skilled in the art using well-known techniques to identify human chromosome 2, as well as the specific locus thereof, that contains the DNA of IL-1 ligand family members. Useful techniques include, but are not limited to, using the sequence or portions, including oligonucleotides, as a probe in various well-known techniques such as radiation hybrid mapping (high resolution), in situ hybridization to chromosome spreads (moderate resolution), and Southern blot hybridization to hybrid cell lines containing individual human chromosomes (low resolution).

For example, chromosomes can be mapped by radiation hybridization. PCR is performed using the Whitehead Institute/MIT Center for Genome Research Genebridge4 panel of 93 radiation hybrids (http://www-genome.wi.mit.edu/ftp/distribution/human_STS_releases/july97/rhmap/genebrid ge4.html). Primers are used which lie within a putative exon of the gene of interest and which amplify a product from human genomic DNA, but do not amplify hamster genomic DNA. The results of the PCRs are converted into a data vector that is submitted to the Whitehead/MIT Radiation Mapping site on the internet (http://www-seq.wi.mit.edu). The data is scored and the chromosomal assignment and placement relative to known Sequence Tag Site (STS) markers on the radiation hybrid map is provided. The following web site provides additional information about radiation hybrid mapping: http://www-genome.wi.mit.edu/ftp/distribution/human_STS_releases/july97/07-97.INTRO.html).

As set forth below, the DNA of SEQ ID NO:1, has been mapped by high-throughput-shotgun sequencing to the 2q11-12 region of human chromosome 2. Human chromosome 2 is associated with specific diseases which include but are not limited to glaucoma, ectodermal dysplasia, insulin-dependent diabetes mellitus, wrinkly skin syndrome, T-cell leukemia/lymphoma, and tibial muscular dystrophy. Thus, the nucleic acids of SEQ ID NO:1, SEQ ID NO:3, or a fragment thereof can be used by one skilled in the art using well-known techniques to analyze abnormalities associated with gene mapping to chromosomes 2. This enables one to distinguish conditions in which this marker is rearranged or deleted. In addition, nucleic acid fragments of SEQ ID NO:1 or a fragment thereof can be used as a positional marker to map other genes of unknown location.

The DNA may be used in developing treatments for any disorder mediated (directly or indirectly) by defective, or insufficient amounts of, the genes corresponding to the nucleic acids of the invention. Disclosure herein of native nucleotide sequences permits the detection of defective genes, and the replacement thereof with normal genes. Defective genes may be detected in in vitro diagnostic assays, and by comparison of a native nucleotide sequence disclosed herein with that of a gene derived from a person suspected of harboring a defect in this gene.

Other useful fragments of the nucleic acids of the present invention include antisense or sense oligonucleotides comprising a single-stranded nucleic acid sequence (either RNA or DNA) capable of binding to target mRNA (sense) or DNA (antisense) sequences.

Antisense RNA and DNA molecules of the present invention act to directly block the translation of mRNA by hybridizing to targeted mRNA and preventing polypeptide translation. Antisense approaches involve the design of oligonucleotides (either DNA or RNA) that are complementary to a FIL-1 theta mRNA. The antisense oligonucleotides will bind to the complementary target gene mRNA transcripts and prevent translation. In accordance with this invention, absolute complementarity, although preferred, is not required. A sequence “complementary” to a portion of a nucleic acid, as referred to herein, means a sequence having sufficient complementarity to be able to hybridize with the nucleic acid, forming a stable duplex (or triplex, as appropriate). In the case of double-stranded antisense nucleic acids, a single strand of the duplex DNA may thus be tested, or triplex formation may be assayed. The ability to hybridize will depend on both the degree of complementarity and the length of the antisense nucleic acid. Preferred oligonucleotides that are complementary to the 5′ end of the message, e.g., the 5′ untranslated sequence up to and including the AUG initiation codon. However, oligonucleotides complementary to the 5′- or 3′-non-translated, non-coding regions of a FIL-1 theta gene transcript, or to the coding regions, are useful in an antisense approach to inhibit translation of endogenous FIL-1 theta mRNA. Oligonucleotides complementary to the 5′ untranslated region of the mRNA preferably include the complement of the AUG start codon. Antisense nucleic acids are at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides. The oligonucleotides can be DNA or RNA or chimeric mixtures or derivatives or modified versions thereof, single-stranded or double-stranded. Chimeric oligonucleotides, oligonucleosides, or mixed oligonucleotides/oligonucleosides of the invention can be of several different types. These include a first type wherein the “gap” segment of nucleotides is positioned between 5′ and 3′ “wing” segments of linked nucleosides and a second “open end” type wherein the “gap” segment is located at either the 3′ or the 5′ terminus of the oligomeric compound (see, e.g., U.S. Pat. No. 5,985,664). Oligonucleotides of the first type are also known in the art as “gapmers” or gapped oligonucleotides. Oligonucleotides of the second type are also known in the art as “hemimers” or “wingmers”. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc Natl Acad Sci U.S.A. 86: 6553-6556; Lemaitre et al., 1987, Proc Natl Acad Sci 84: 648-652; PCT Publication No. WO88/09810), or hybridization-triggered cleavage agents or intercalating agents. (See, e.g., Zon, 1988, Pharm. Res. 5: 539-549). The antisense molecules should be delivered to cells which express the FIL-1 theta transcript in vivo. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous FIL-1 theta gene transcripts and thereby prevent translation of the FIL-1 theta mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Ribozyme molecules designed to catalytically cleave FIL-1 theta mRNA transcripts can also be used to prevent translation of FIL-1 theta mRNA and expression of FIL-1 theta polypeptides. (See, e.g., PCT International Publication WO90/11364, published Oct. 4, 1990; U.S. Pat. No. 5,824,519). The ribozymes that can be used in the present invention include hammerhead ribozymes (Haseloff and Gerlach, 1988, Nature, 334:585-591), RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (International Patent Application No. WO 88/04300; Been and Cech, 1986, Cell, 47:207-216). As in the antisense approach, the ribozymes can be composed of modified oligonucleotides (e.g. for improved stability, targeting, etc.) and should be delivered to cells which express the FIL-1 theta polypeptide in vivo. A preferred method of delivery involves using a DNA construct “encoding” the ribozyme under the control of a strong constitutive pol III or pol II promoter, so that transfected cells will produce sufficient quantities of the ribozyme to destroy endogenous FIL-1 theta messages and inhibit translation. Because ribozymes, unlike antisense molecules, are catalytic, a lower intracellular concentration is required for efficiency.

Alternatively, endogenous FIL-1 theta gene expression can be reduced by targeting deoxyribonucleotide sequences complementary to the regulatory region of the target gene (i.e., the target gene promoter and/or enhancers) to form triple helical structures that prevent transcription of the target FIL-1 theta gene. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Anti-sense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Oligonucleotides can be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., 1988, Nucl. Acids Res. 16:3209. Methylphosphonate oligonucleotides can be prepared by use of controlled pore glass polymer supports (Sarin et al., 1988, Proc. Natl. Acad. Sci. U.S.A. 85:7448-7451). Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Endogenous target gene expression can also be reduced by inactivating or “knocking out” the target gene or its promoter using targeted homologous recombination (e.g., see Smithies, et al., 1985, Nature 317, 230-234; Thomas and Capecchi, 1987, Cell 51, 503-512; Thompson, et al., 1989, Cell 5, 313-321). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra), or in model organisms such as Caenorhabditis elegans where the “RNA interference” (“RNAi”) technique (Grishok A, Tabara H, and Mello C C, 2000, Genetic requirements for inheritance of RNAi in C. elegans, Science 287 (5462): 2494-2497), or the introduction of transgenes (Dernburg A F, Zalevsky J, Colaiacovo M P, and Villeneuve A M, 2000, Transgene-mediated cosuppression in the C. elegans germ line, Genes Dev. 14 (13): 1578-1583) are used to inhibit the expression of specific target genes. However this approach can be adapted for use in humans provided the recombinant DNA constructs are directly administered or targeted to the required site in vivo using appropriate vectors such as viral vectors.

Organisms that have enhanced, reduced, or modified expression of the gene(s) corresponding to the nucleic acid sequences disclosed herein are provided. The desired change in gene expression can be achieved through the use of antisense nucleic acids or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39). Transgenic animals that have multiple copies of the gene(s) corresponding to the nucleic acid sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1). In addition, organisms are provided in which the gene(s) corresponding to the nucleic acid sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc. Natl. Acad. Sci. USA 90(16): 7431-7435; Clark et al., 1994, Proc. Natl. Acad. Sci. USA 91(2): 719-722), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523). These organisms with altered gene expression are preferably eukaryotes and more preferably are mammals. Such organisms are useful for the development of non-human models for the study of disorders involving the corresponding gene(s), and for the development of assay systems for the identification of molecules that interact with the polypeptide product(s) of the corresponding gene(s).

Use of FIL-1 Theta Polypeptides and Fragments

The polypeptides and fragments of polypeptides described herein find many uses that include, but are not limited to, the following:

Purifying proteins and measuring activity thereof

Delivery Agents

Therapeutic and Research Reagents

Controls for peptide fragmentation

Identification of unknown proteins

Identification of compounds that alter activity

Preparation of Antibodies

Each polypeptide of the present invention finds use as a protein purification reagent. The polypeptides may be attached to a solid support material and used to purify binding partner proteins by affinity chromatography. In particular embodiments, a polypeptide (in any form described herein that is capable of binding a binding partner) is attached to a solid support by conventional procedures. As one example, chromatography columns containing functional groups that will react with functional groups on amino acid side chains of proteins are available (Pharmacia Biotech, Inc., Piscataway, N.J.). In an alternative, a polypeptide/Fc protein (as discussed above) is attached to Protein A- or Protein G-containing chromatography columns through interaction with the Fc moiety.

The polypeptide also finds use in purifying or identifying cells that express the binding partner on the cell surface. Polypeptides are bound to a solid phase such as a column chromatography matrix or a similar suitable substrate. For example, magnetic microspheres can be coated with the polypeptides and held in an incubation vessel through a magnetic field. Suspensions of cell mixtures containing the binding partner expressing cells are contacted with the solid phase having the polypeptides thereon. Cells expressing the binding partner on the cell surface bind to the fixed polypeptides, and unbound cells then are washed away.

Alternatively, the polypeptides can be conjugated to a detectable moiety, then incubated with cells to be tested for binding partner expression. After incubation, unbound labeled matter is removed and the presence or absence of the detectable moiety on the cells is determined.

In a further alternative, mixtures of cells suspected of containing cells expressing the binding partner are incubated with biotinylated polypeptides. Incubation periods are typically at least one hour in duration to ensure sufficient binding. The resulting mixture then is passed through a column packed with avidin-coated beads, whereby the high affinity of biotin for avidin provides binding of the desired cells to the beads. Procedures for using avidin-coated beads are known (Berenson et al., J. Cell. Biochem., 10D:239, 1986). Washing to remove unbound material, and the release of the bound cells, are performed using conventional methods.

Polypeptides of this invention also find use in measuring the biological activity of the binding partner protein in terms of their binding affinity. Polypeptides thus may be employed by those conducting “quality assurance” studies, e.g., to monitor shelf life and stability of protein under different conditions. Accordingly, polypeptides described herein are useful in a binding affinity studies to measure the biological activity of a binding partner protein that has been stored at different temperatures, or produced in different cell types. Polypeptides of the invention may be used to determine whether biological activity is retained after modification of a binding partner protein (e.g., chemical modification, truncation, mutation, etc.). The binding affinity of the modified binding partner protein is compared to that of an unmodified binding partner protein to detect any adverse impact of the modifications on biological activity of the binding partner. The biological activity of a binding partner protein thus can be ascertained before it is used in a research study, for example.

Polypeptides described herein also find use as carriers in methods for delivering diagnostic or therapeutic agents attached thereto to cells bearing the binding partner of the polypeptides. The polypeptides thus can be used to deliver diagnostic or therapeutic agents to such cells (or to other cell types found to express the binding partner on the cell surface) in in vitro or in vivo procedures.

Detectable (diagnostic) and therapeutic agents that may be attached to a polypeptide include, but are not limited to, toxins, other cytotoxic agents, drugs, radionuclides, chromophores, enzymes that catalyze a colorimetric or fluorometric reaction, and the like, with the particular agent being chosen according to the intended application. Among the toxins are ricin, abrin, diphtheria toxin, Pseudomonas aeruginosa exotoxin A, ribosomal inactivating proteins, mycotoxins such as trichothecenes, and derivatives and fragments (e.g., single chains) thereof. Radionuclides suitable for diagnostic use include, but are not limited to, ¹²³I, ¹³¹I, ^{99m}Tc, ¹¹¹In, and ⁷⁶Br. Examples of radionuclides suitable for therapeutic use are ¹³¹I, ²¹¹At, ⁷⁷Br, ¹⁸⁶Re, ¹⁸⁸Re, ²¹²Pb, ²¹²Bi, ¹⁰⁹Pd, ⁶⁴Cu, and ⁶⁷Cu.

Such agents may be attached to the polypeptide by any suitable conventional procedure. The polypeptide comprises functional groups on amino acid side chains that can be reacted with functional groups on a desired agent to form covalent bonds, for example. Alternatively, the protein or agent may be derivatized to generate or attach a desired reactive functional group. The derivatization may involve attachment of one of the bifunctional coupling reagents available for attaching various molecules to proteins (Pierce Chemical Company, Rockford, Ill.). A number of techniques for radiolabeling proteins are known. Radionuclide metals may be attached to polypeptides by using a suitable bifunctional chelating agent, for example.

Conjugates comprising polypeptides and a suitable diagnostic or therapeutic agent (preferably covalently linked) are thus prepared. The conjugates are administered or otherwise employed in an amount appropriate for the particular application.

Polypeptides of the invention and polypeptide variant of this invention may be therapeutics for disorders mediated (directly or indirectly) by defective, or insufficient amounts of the polypeptides. These polypeptides may be administered to a mammal afflicted with such a disorder.

The polypeptides may also be employed in inhibiting a biological activity of the binding partner, in in vitro or in vivo procedures. For example, a purified FIL-1 theta polypeptide, fragment, or variant of SEQ ID NO:2 or polypeptide fragment or variant of SEQ ID NO:4, or a fragments of SEQ ID NO:15, can be used to inhibit binding of endogenous FIL-1 theta to its cell surface receptor. Biological effects that result from the binding of FIL-1 theta to its endogenous receptors or receptor are inhibited by blocking the binding and subsequence signal transduction.

Polypeptides of the invention may be administered to a mammal to treat a disorder mediated by one or more binding partners of the polypeptide. Such binding partner-mediated disorders include conditions caused (directly or indirectly) or exacerbated by the binding partner.

Compositions of the present invention may contain a polypeptide in any form described herein, such as native proteins, variants, derivatives, oligomers, and biologically active fragments. In particular embodiments, the composition comprises a soluble polypeptide or an oligomer comprising soluble polypeptides of the invention.

For purposes of this disclosure, the terms “illness,” “disease,” “medical condition,” “abnormal condition” and the like are used interchangeably with the term “medical disorder.” The terms “treat”, “treating”, and “treatment” used herein include curative, preventative (e.g., prophylactic) and palliative or ameliorative treatment. For such therapeutic uses, FIL-1 theta polypeptides and fragments, FIL-1 theta nucleic acids encoding the FIL-1 theta family polypeptides, and/or agonists or antagonists of the FIL-1 theta polypeptide such as antibodies can be administered to the patient in need through well-known means. Compositions of the present invention can contain a polypeptide in any form described herein, such as native polypeptides, variants, derivatives, oligomers, and biologically active fragments. In particular embodiments, the composition comprises a soluble polypeptide or an oligomer comprising soluble FIL-1 theta polypeptides.

In practicing the method of treatment or use of the present invention, a therapeutically effective amount of a therapeutic agent of the present invention is administered to a patient having a condition to be treated, preferably to treat or ameliorate diseases associated with the activity of a FIL-1 theta or IL-1 family polypeptide. “Therapeutic agent” includes without limitation any of the FIL-1 theta polypeptides, fragments, and variants; nucleic acids encoding the FIL-1 theta polypeptides, fragments, and variants; agonists or antagonists of the FIL-1 theta polypeptides such as antibodies; FIL-1 theta polypeptide binding partners; complexes formed from the FIL-1 theta polypeptides, fragments, variants, and binding partners, etc. As used herein, the term “therapeutically effective amount” means the total amount of each therapeutic agent or other active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual therapeutic agent or active ingredient, administered alone, the term refers to that ingredient alone. When applied to a combination, the term refers to combined amounts of the ingredients that result in the therapeutic effect, whether administered in combination, serially or simultaneously. As used herein, the phrase “administering a therapeutically effective amount” of a therapeutic agent means that the patient is treated with said therapeutic agent in an amount and for a time sufficient to induce an improvement, and preferably a sustained improvement, in at least one indicator that reflects the severity of the disorder. An improvement is considered “sustained” if the patient exhibits the improvement on at least two occasions separated by one or more weeks. The degree of improvement is determined based on signs or symptoms, and determinations may also employ questionnaires that are administered to the patient, such as quality-of-life questionnaires. Various indicators that reflect the extent of the patient's illness may be assessed for determining whether the amount and time of the treatment is sufficient. The baseline value for the chosen indicator or indicators is established by examination of the patient prior to administration of the first dose of the therapeutic agent. Preferably, the baseline examination is done within about 60 days of administering the first dose. If the therapeutic agent is being administered to treat acute symptoms, the first dose is administered as soon as practically possible after the injury has occurred. Improvement is induced by administering therapeutic agents such as FIL-1 theta polypeptides or antagonists until the patient manifests an improvement over baseline for the chosen indicator or indicators. In treating chronic conditions, this degree of improvement is obtained by repeatedly administering this medicament over a period of at least a month or more, e.g., for one, two, or three months or longer, or indefinitely. A period of one to six weeks, or even a single dose, often is sufficient for treating acute conditions. For injuries or acute conditions, a single dose may be sufficient. Although the extent of the patient's illness after treatment may appear improved according to one or more indicators, treatment may be continued indefinitely at the same level or at a reduced dose or frequency. Once treatment has been reduced or discontinued, it later may be resumed at the original level if symptoms should reappear.

Dosing. One skilled in the pertinent art will recognize that suitable dosages will vary, depending upon such factors as the nature and severity of the disorder to be treated, the patient's body weight, age, general condition, and prior illnesses and/or treatments, and the route of administration. Preliminary doses can be determined according to animal tests, and the scaling of dosages for human administration is performed according to art-accepted practices such as standard dosing trials. For example, the therapeutically effective dose can be estimated initially from cell culture assays. The dosage will depend on the specific activity of the compound and can be readily determined by routine experimentation. A dose may be formulated in animal models to achieve a circulating plasma concentration range that includes the IC50 (i.e., the concentration of the test compound which achieves a half-maximal inhibition of symptoms) as determined in cell culture, while minimizing toxicities. Such information can be used to more accurately determine useful doses in humans. Ultimately, the attending physician will decide the amount of polypeptide of the present invention with which to treat each individual patient. Initially, the attending physician will administer low doses of polypeptide of the present invention and observe the patient's response. Larger doses of polypeptide of the present invention may be administered until the optimal therapeutic effect is obtained for the patient, and at that point the dosage is not increased further. It is contemplated that the various pharmaceutical compositions used to practice the method of the present invention should contain about 0.01 ng to about 100 mg (preferably about 0.1 ng to about 10 mg, more preferably about 0.1 microgram to about 1 mg) of polypeptide of the present invention per kg body weight. In one embodiment of the invention, FIL-1 theta polypeptides or antagonists are administered one time per week to treat the various medical disorders disclosed herein, in another embodiment is administered at least two times per week, and in another embodiment is administered at least three times per week. If injected, the effective amount of FIL-1 theta polypeptides or antagonists per adult dose ranges from 1-20 mg/m², and preferably is about 5-12 mg/m². Alternatively, a flat dose may be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, the various indications described below are treated by administering a preparation acceptable for injection containing FIL-1 theta polypeptides or antagonists at 25 mg/dose, or alternatively, containing 50 mg per dose. The 25 mg or 50 mg dose may be administered repeatedly, particularly for chronic conditions. If a route of administration other than injection is used, the dose is appropriately adjusted in accord with standard medical practices. In many instances, an improvement in a patient's condition will be obtained by injecting a dose of about 25 mg of FIL-1 theta polypeptides or antagonists one to three times per week over a period of at least three weeks, or a dose of 50 mg of FIL-1 theta polypeptides or antagonists one or two times per week for at least three weeks, though treatment for longer periods may be necessary to induce the desired degree of improvement. For incurable chronic conditions, the regimen may be continued indefinitely, with adjustments being made to dose and frequency if such are deemed necessary by the patient's physician. The foregoing doses are examples for an adult patient who is a person who is 18 years of age or older. For pediatric patients (age 4-17), a suitable regimen involves the subcutaneous injection of 0.4 mg/kg, up to a maximum dose of 25 mg of FIL-1 theta polypeptides or antagonists, administered by subcutaneous injection one or more times per week. If an antibody against a FIL-1 theta polypeptide is used as the FIL-1 theta polypeptide antagonist, a preferred dose range is 0.1 to 20 mg/kg, and more preferably is 1-10 mg/kg. Another preferred dose range for an anti-FIL-1 theta polypeptide antibody is 0.75 to 7.5 mg/kg of body weight. Humanized antibodies are preferred, that is, antibodies in which only the antigen-binding portion of the antibody molecule is derived from a non-human source. Such antibodies may be injected or administered intravenously.

Formulations. Compositions comprising an effective amount of a FIL-1 theta polypeptide of the present invention (from whatever source derived, including without limitation from recombinant and non-recombinant sources), in combination with other components such as a physiologically acceptable diluent, carrier, or excipient, are provided herein. The term “pharmaceutically acceptable” means a non-toxic material that does not interfere with the effectiveness of the biological activity of the active ingredient(s). Formulations suitable for administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The polypeptides can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323. Such compositions will influence the physical state, solubility, stability, rate of in vivo release, and rate of in vivo clearance, and are thus chosen according to the intended application, so that the characteristics of the carrier will depend on the selected route of administration. In one preferred embodiment of the invention, sustained-release forms of FIL-1 theta polypeptides are used. Sustained-release forms suitable for use in the disclosed methods include, but are not limited to, FIL-1 theta polypeptides that are encapsulated in a slowly-dissolving biocompatible polymer (such as the alginate microparticles described in U.S. Pat. No. 6,036,978), admixed with such a polymer (including topically applied hydrogels), and or encased in a biocompatible semi-permeable implant.

Combinations of Therapeutic Compounds. A FIL-1 theta polypeptide of the present invention may be active in multimers (e.g., heterodimers or homodimers) or complexes with itself or other polypeptides. As a result, pharmaceutical compositions of the invention may comprise a polypeptide of the invention in such multimeric or complexed form. The pharmaceutical composition of the invention may be in the form of a complex of the polypeptide(s) of present invention along with polypeptide or peptide antigens. The invention further includes the administration of FIL-1 theta polypeptides or antagonists concurrently with one or more other drugs that are administered to the same patient in combination with the FIL-1 theta polypeptides or antagonists, each drug being administered according to a regimen suitable for that medicament. “Concurrent administration” encompasses simultaneous or sequential treatment with the components of the combination, as well as regimens in which the drugs are alternated, or wherein one component is administered long-term and the other(s) are administered intermittently. Components may be administered in the same or in separate compositions, and by the same or different routes of administration. Examples of components that may be included in the pharmaceutical composition of the invention are: cytokines, lymphokines, or other hematopoietic factors such as M-CSF, GM-CSF, TNF, IL-1, IL-2, IL-3, IL4, IL-5, IL-6, IL-7, IL-8, IL-9, IL-10, IL-11, IL-12, IL-13, IL-14, IL-15, IL-17, IL-18, IFN, TNFO, TNF1, TNF2, G-CSF, Meg-CSF, thrombopoietin, stem cell factor, and erythropoietin. The pharmaceutical composition may further contain other agents which either enhance the activity of the polypeptide or compliment its activity or use in treatment. Such additional factors and/or agents may be included in the pharmaceutical composition to produce a synergistic effect with polypeptide of the invention, or to minimize side effects. Conversely, a FIL-1 theta polypeptide or antagonist of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent. Additional examples of drugs to be administered concurrently include but are not limited to antivirals, antibiotics, analgesics, corticosteroids, antagonists of inflammatory cytokines, non-steroidal anti-inflammatories, pentoxifylline, thalidomide, and disease-modifying antirheumatic drugs (DMARDs) such as azathioprine, cyclophosphamide, cyclosporine, hydroxychloroquine sulfate, methotrexate, leflunomide, minocycline, penicillamine, sulfasalazine and gold compounds such as oral gold, gold sodium thiomalate, and aurothioglucose. Additionally, FIL-1 theta polypeptides or antagonists may be combined with a second FIL-1 theta polypeptide/antagonist, including an antibody against a FIL-1 theta polypeptide, or a FIL-1 theta polypeptide-derived peptide that acts as a competitive inhibitor of a native FIL-1 theta polypeptide.

Routes of Administration. Any efficacious route of administration may be used to therapeutically administer FIL-1 theta polypeptides or antagonists thereof, including those compositions comprising nucleic acids. Parenteral administration includes injection, for example, via intra-articular, intravenous, intramuscular, intralesional, intraperitoneal or subcutaneous routes by bolus injection or by continuous infusion., and also includes localized administration, e.g., at a site of disease or injury. Other suitable means of administration include sustained release from implants; aerosol inhalation and/or insufflation.; eyedrops; vaginal or rectal suppositories; buccal preparations; oral preparations, including pills, syrups, lozenges or chewing gum; and topical preparations such as lotions, gels, sprays, ointments or other suitable techniques. Alternatively, polypeptideaceous FIL-1 theta polypeptides or antagonists may be administered by implanting cultured cells that express the polypeptide, for example, by implanting cells that express FIL-1 theta polypeptides or antagonists. Cells may also be cultured ex vivo in the presence of polypeptides of the present invention in order to proliferate or to produce a desired effect on or activity in such cells. Treated cells can then be introduced in vivo for therapeutic purposes. In another embodiment, the patient's own cells are induced to produce FIL-1 theta polypeptides or antagonists by transfection in vivo or ex vivo with a DNA that encodes FIL-1 theta polypeptides or antagonists. This DNA can be introduced into the patient's cells, for example, by injecting naked DNA or liposome-encapsulated DNA that encodes FIL-1 theta polypeptides or antagonists, or by other means of transfection. Nucleic acids of the invention may also be administered to patients by other known methods for introduction of nucleic acid into a cell or organism (including, without limitation, in the form of viral vectors or naked DNA). When FIL-1 theta polypeptides or antagonists are administered in combination with one or more other biologically active compounds, these may be administered by the same or by different routes, and may be administered simultaneously, separately or sequentially.

Oral Administration. When a therapeutically effective amount of polypeptide of the present invention is administered orally, polypeptide of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% polypeptide of the present invention, and preferably from about 25 to 90% polypeptide of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of polypeptide of the present invention, and preferably from about 1 to 50% polypeptide of the present invention. When a therapeutically effective amount of polypeptide of the present invention is administered by intravenous, cutaneous or subcutaneous injection, polypeptide of the present invention will be in the form of a pyrogen-free, parenterally acceptable aqueous solution. The preparation of such parenterally acceptable polypeptide solutions, having due regard to pH, isotonicity, stability, and the like, is within the skill in the art. A preferred pharmaceutical composition for intravenous, cutaneous, or subcutaneous injection should contain, in addition to polypeptide of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. The duration of intravenous therapy using the pharmaceutical composition of the present invention will vary, depending on the severity of the disease being treated and the condition and potential idiosyncratic response of each individual patient. It is contemplated that the duration of each application of the polypeptide of the present invention will be in the range of 12 to 24 hours of continuous intravenous administration. Ultimately the attending physician will decide on the appropriate duration of intravenous therapy using the pharmaceutical composition of the present invention.

Bone and Tissue Administration. For compositions of the present invention which are useful for bone, cartilage, tendon or ligament disorders, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a polypeptide of the invention which may also optionally be included in the composition as described above, may alternatively or additionally, be administered simultaneously or sequentially with the composition in the methods of the invention. Preferably for bone and/or cartilage formation, the composition would include a matrix capable of delivering the polypeptide-containing composition to the site of bone and/or cartilage damage, providing a structure for the developing bone and cartilage and optimally capable of being resorbed into the body. Such matrices may be formed of materials presently in use for other implanted medical applications. The choice of matrix material is based on biocompatibility, biodegradability, mechanical properties, cosmetic appearance and interface properties. The particular application of the compositions will define the appropriate formulation. Potential matrices for the compositions may be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure polypeptides or extracellular matrix components. Other potential matrices are nonbiodegradable and chemically defined, such as sintered hydroxapatite, bioglass, aluminates, or other ceramics Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability. Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the polypeptide compositions from disassociating from the matrix. A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethyl-cellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorption of the polypeptide from the polymer matrix and to provide appropriate handling of the composition, yet not so much that the progenitor cells are prevented from infiltrating the matrix, thereby providing the polypeptide the opportunity to assist the osteogenic activity of the progenitor cells. In further compositions, polypeptides of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-alpha and TGF-beta), and insulin-like growth factor (IGF). The therapeutic compositions are also presently valuable for veterinary applications. Particularly domestic animals and thoroughbred horses, in addition to humans, are desired patients for such treatment with polypeptides of the present invention. The dosage regimen of a polypeptide-containing pharmaceutical composition to be used in tissue regeneration will be determined by the attending physician considering various factors which modify the action of the polypeptides, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage may vary with the type of matrix used in the reconstitution and with inclusion of other polypeptides in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Veterinary Uses. In addition to human patients, FIL-1 theta polypeptides and antagonists are useful in the treatment of disease conditions in non-human animals, such as pets (dogs, cats, birds, primates, etc.), domestic farm animals (horses cattle, sheep, pigs, birds, etc.), or any animal that suffers from a TNFα-mediated inflammatory or arthritic condition. In such instances, an appropriate dose may be determined according to the animal's body weight. For example, a dose of 0.2-1 mg/kg may be used. Alternatively, the dose is determined according to the animal's surface area, an exemplary dose ranging from 0.1-20 mg/m², or more preferably, from 5-12 mg/m². For small animals, such as dogs or cats, a suitable dose is 0.4 mg/kg. In a preferred embodiment, FIL-1 theta polypeptides or antagonists (preferably constructed from genes derived from the same species as the patient), is administered by injection or other suitable route one or more times per week until the animal's condition is improved, or it may be administered indefinitely.

Manufacture of Medicaments. The present invention also relates to the use FIL-1 theta polypeptides, fragments, and variants; nucleic acids encoding the FIL-1 theta family polypeptides, fragments, and variants; agonists or antagonists of the FIL-1 theta polypeptides such as antibodies; FIL-1 theta polypeptide binding partners; complexes formed from the FIL-1 theta family polypeptides, fragments, variants, and binding partners, etc, in the manufacture of a medicament for the prevention or therapeutic treatment of each medical disorder disclosed herein.

Compositions comprising an effective amount of a polypeptide of the present invention, in combination with other components such as a physiologically acceptable diluent, carrier, or excipient, are provided herein. The polypeptides can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed., Mack Publishing Company, Easton, Pa., 1980.

In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance, and are thus chosen according to the intended application.

The compositions of the invention can be administered in any suitable manner, e.g., topically, parenterally, or by inhalation. The term “parenteral” includes injection, e.g., by subcutaneous, intravenous, or intramuscular routes, also including localized administration, e.g., at a site of disease or injury. Sustained release from implants is also contemplated. One skilled in the pertinent art will recognize that suitable dosages will vary, depending upon such factors as the nature of the disorder to be treated, the patient's body weight, age, and general condition, and the route of administration.

Preliminary doses can be determined according to animal tests, and the scaling of dosages for human administration is performed according to art-accepted practices.

Compositions comprising nucleic acids in physiologically acceptable formulations are also contemplated. DNA may be formulated for injection, for example.

Another use of the polypeptide of the present invention is as a research tool for studying the biological effects that result from the interactions of FIL-1 theta, with its binding partner, or from inhibiting these interactions, on different cell types. Polypeptides also may be employed in in vitro assays for detecting FIL-1 theta, the respective binding partners or the interactions thereof.

Another embodiment of the invention relates to uses of the polypeptides of the invention to study cell signal transduction. IL-1 family ligands play a central role in protection against infection and immune inflammatory responses which includes cellular signal transduction, activating vascular endothelial cells and lymphocytes, induction of inflammatory cytokines, acute phase proteins, hematopoiesis, fever, bone resorption, prostaglandins, metalloproteinases, and adhesion molecules. With the continued increase in the number of known IL-1 family members, a suitable classification scheme is one based on comparing polypeptide structure as well as function (activation and regulatory properties). Thus, FIL-1 theta, like other IL-1 family ligands (IL-1α, IL-1β, and IL-18), is likely involved in many of the functions noted above and involve in promoting inflammatory responses. Accordingly, FIL-1 theta is likely a factor in the causation and maintenance of inflammatory and/or autoimmune diseases such as rheumatoid arthritis, inflammatory bowel disease, and psoriasis. As such, alterations in the expression and/or activation of the polypeptides of the invention can have profound effects on a plethora of cellular processes, including, but not limited to, activation or inhibition of cell specific responses and proliferation. Expression of cloned FIL-1 theta, or functionally inactive mutants thereof can be used to identify the role a particular protein plays in mediating specific signaling events.

Cellular signaling often involves a molecular activation cascade, during which a receptor propagates a ligand-receptor mediated signal by specifically activating intracellular kinases that phosphorylate target substrates. These substrates can themselves be kinases which become activated following phosphorylation. Alternatively, they can be adaptor molecules that facilitate down stream signaling through protein-protein interaction following phosphorylation. Regardless of the nature of the substrate molecule(s), expressed functionally active versions of FIL-1 theta, and their binding partners can be used to identify what substrate(s) were recognized and activated by the polypeptides of the invention. As such, these novel polypeptides can be used as reagents to identify novel molecules involved in signal transduction pathways.

The polypeptides of the present invention find use in screening assays to identify compounds and small molecules which inhibit (antagonize) or enhance (agonize) activation of the polypeptides of the instant invention. Thus, for example, polypeptides described herein may be used to identify antagonists and agonists from cells, cell-free preparations, chemical libraries, and natural product mixtures. The antagonists and agonists may be natural or modified substrates, ligands, enzymes, receptors, etc. of the polypeptides of the instant invention, or may be structural or functional mimetics of the polypeptides. Potential antagonists of the polypeptides of the instant invention may include small molecules, peptides, and antibodies that bind to and occupy a binding site of the polypeptides, causing the binding sites to be unavailable for binding to their ligands and therefore preventing normal biological activity. Other potential antagonists are antisense molecules that may hybridize to mRNA in vivo and block translation of the mRNA into the polypeptides of the instant invention. Potential agonists include small molecules, peptides and antibodies that bind to the instant polypeptides and elicit the same or enhanced biological effects as those caused by the binding of the polypeptides of the instant invention.

Typically, small molecule agonists and antagonists have molecular weights of less than 10K and may possess a number of physiochemical and pharmacological properties that enhance cell penetration, resist degradation and prolong their physiological half-lives. (Gibbs, J., Pharmaceutical Research in Molecular Oncology, Cell, Vol. 79 (1994).) Antibodies, which include intact molecules as well as fragments such as Fab and F(ab′)2 fragments, may be used to bind to and inhibit the polypeptides of the instant invention by blocking the commencement of a signaling cascade. It is preferable that the antibodies are humanized, and more preferable that the antibodies are human. The antibodies of the present invention may be prepared by any of a variety of well-known methods In general the screening methods of the present invention are performed to identify compounds that alter an activity associated with FIL-1 theta by a) mixing a test compound which is a potential agonist or antagonist with a FIL-1 theta polypeptide of the invention; and, b) determining whether the test compound alters FIL-1 theta activity of the polypeptide. Identifying compounds that inhibit the binding activity of a polypeptides of this invention includes a) mixing a test compound with the polypeptide of this invention, and a binding partner of the polypeptide; and, b) determining whether the test compound inhibits the binding activity of the polypeptide.

A number of screening techniques that include the use of cell based or in vitro based specific binding assays or tests are known. (See, for example, High Throughput Screening: The Discovery of Bioactive Substances, John P. Devlin (ed.), Marcel Dekker, New York, 1997, ISBN: 0-8247-0067-8., http://www.lab-robotics.org/,http://www.sbsonline.org/, all of which are incorporated herein by reference) When combined with integrated robotic systems, high throughput screening techniques can be used to test and screen large collections of chemical compounds and/or natural products for antagonist or agonist activity within a short amount of time. Specific assays or tests include homogeneous assay formats such as fluorescence resonance energy transfer, time resolved fluorescence resonance energy transfer, fluorescence polarization, scintillation proximity assays, reporter gene assays, fluorescence quenched enzyme substrate, chromogenic enzyme substrate and electrochemiluminesence, as well as, more traditional heterogeneous assay formats such as enzyme linked immunosorbant assays (ELISA) or radioimmunoassays. Homogeneous assays are mix and read style assays that are very amenable to robotic application, whereas heterogeneous assays require separation of bound analyte from free analyte by more complex unit operations such as filtration, centrifugation or washing. These assays can be utilized to detect a wide variety of specific biomolecular interactions and the inhibition of specific biomolecular interactions by small organic molecules, drug candidates, antibodies, peptides and other antagonists and/or agonists. Specific biomolecular interactions include, but are not limited to, protein-protein interactions, receptor-ligand interactions, enzyme-substrate interactions, etc.

One embodiment of a method for identifying molecules which antagonize or inhibit polypeptides of the present invention involves adding a candidate molecule to a medium which contains cells that express the polypeptides of the instant invention; changing the conditions of the medium so that, but for the presence of the candidate molecule, the polypeptides would be bound to their ligands; and observing the binding and stimulation or inhibition of a functional response. The activity of the cells that were contacted with the candidate molecule may then be compared with the identical cells that were not contacted with the candidate molecule and agonists and antagonists of the polypeptides of the instant invention may be identified. The measurement of biological activity may be performed by a number of well-known methods such as measuring the amount of protein present (e.g. an ELISA) or of the protein's activity. A decrease in biological stimulation or activation would indicate an antagonist. An increase would indicate an agonist. Specifically, one embodiment of the instant invention includes agonists and antagonists of FIL-1 theta.

Screening assays can further be designed to find molecules that mimic the biological activity of the polypeptides of the instant invention. Molecules which mimic the biological activity of a polypeptide may be useful for enhancing the biological activity of the polypeptide. To identify compounds for therapeutically active agents that mimic the biological activity of a polypeptide, it must first be determined whether a candidate molecule binds to the polypeptide. A binding candidate molecule is then added to a biological assay to determine its biological effects. The biological effects of the candidate molecule are then compared to those of the polypeptide.

A polypeptide or peptide fingerprint can be entered into or compared to a database of known proteins to assist in the identification of the unknown protein using mass spectrometry (W. J. Henzel et al., Proc. Natl. Acad. Sci. USA, 90:5011-5015, 1993; Fenyo et al., Electrophoresis, 19:998-1005, 1998). A variety of computer software programs to facilitate these comparisons are accessible via the Internet, such as Protein Prospector (Internet site: prospector.uscf.edu), MultiIdent (Internet site: www.expasy.ch/sprot/multiident.html), PeptideSearch (Internet site:www.mann.embl-heiedelberg.dedeSearch/FR_PeptideSearch Form.html), and ProFound (Internet site:www.chait-sgi.rockefeller.edu/cgi-bin/prot-id-frag.html). These programs allow the user to specify the cleavage agent and the molecular weights of the fragmented peptides within a designated tolerance. The programs compare observed molecular weights to predicted peptide molecular weights derived from sequence databases to assist in determining the identity of the unknown protein.

In addition, a polypeptide or peptide digest can be sequenced using tandem mass spectrometry (MS/MS) and the resulting sequence searched against databases (Eng et al., J. Am. Soc. Mass Spec., 5:976-989, 1994; M. Mann et al., Anal. Chem., 66:4390-4399, 1994; and J. A. Taylor et al., Rapid Comm. Mass Spec., 11:1067-1075, 1997). Searching programs that can be used in this process exist on the Internet, such as Lutefisk 97 (Internet site: www.lsbc.com:70/Lutefisk97.html), and the Protein Prospector, Peptide Search and ProFound programs described above.

Therefore, adding the sequence of a gene and its predicted protein sequence and peptide fragments to a sequence database can aid in the identification of unknown proteins using mass spectrometry.

Antibodies that are immunoreactive with the polypeptides of the invention are provided herein. Such antibodies specifically bind to the polypeptides via the antigen-binding sites of the antibody (as opposed to non-specific binding). Thus, the polypeptides of SEQ ID NO:2 and SEQ ID NO:4, fragments, variants, fusion proteins, etc., as set forth above may be employed as “immunogens” in producing antibodies immunoreactive therewith. More specifically, the polypeptides, fragment, variants, fusion proteins, etc. contain antigenic determinants or epitopes that elicit the formation of antibodies.

These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon protein folding (C. A. Janeway, Jr. and P. Travers, Immuno Biology, 3:9, Garland Publishing Inc., 2nd ed., 1996). Because folded proteins have complex surfaces, the number of epitopes available is quite numerous; however, due to the conformation of the protein and steric hindrances, the number of antibodies that actually bind to the epitopes is less than the number of available epitopes (C. A. Janeway, Jr. and P. Travers, Immuno Biology, 2:14, Garland Publishing Inc., 2nd ed., 1996). Epitopes may be identified by any of the methods known in the art.

Thus, one aspect of the present invention relates to the antigenic epitopes of the polypeptides of the invention. Such epitopes are useful for raising antibodies, in particular monoclonal antibodies, as described in more detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

As to the antibodies that can be elicited by the epitopes of the polypeptides of the invention, whether the epitopes have been isolated or remain part of the polypeptides, both polyclonal and monoclonal antibodies may be prepared by conventional techniques. See, for example, Kennet et al. (eds.), Monoclonal Antibodies, Hybridomas: A New Dimension in Biological Analyses, Plenum Press, New York, 1980; and Harlow and Land (eds.), Antibodies: A Laboratory Manual, Cold Spring Harbor Laboratory Press, Cold Spring Harbor, N.Y., 1988.

Hybridoma cell lines that produce monoclonal antibodies specific for the polypeptides of the invention are also contemplated herein. Such hybridomas may be produced and identified by conventional techniques. One method for producing such a hybridoma cell line comprises immunizing an animal with a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. The monoclonal antibodies may be recovered by conventional techniques.

The monoclonal antibodies of the present invention include chimeric antibodies, e.g., humanized versions of murine monoclonal antibodies. Such humanized antibodies may be prepared by known techniques and offer the advantage of reduced immunogenicity when the antibodies are administered to humans. In one embodiment, a humanized monoclonal antibody comprises the variable region of a murine antibody (or just the antigen-binding site thereof) and a constant region derived from a human antibody. Alternatively, a humanized antibody fragment may comprise the antigen binding site of a murine monoclonal antibody and a variable region fragment (lacking the antigen-binding site) derived from a human antibody. Procedures for the production of chimeric and further engineered monoclonal antibodies include those described in Riechmann et al., Nature, 332:323, 1988; Liu et al., PNAS, 84:3439, 1987; Larrick et al., Bio/Technology, 7:934, 1989; and Winter et al., TIPS, 14:139, 1993. Procedures to generate antibodies transgenically can be found in GB 2,272,440, U.S. Pat. Nos. 5,569,825 and 5,545,806 and related patents claiming priority therefrom, all of which are incorporated by reference herein.

Antigen-binding fragments of the antibodies, which may be produced by conventional techniques, are also encompassed by the present invention. Examples of such fragments include, but are not limited to, Fab and F(ab′)₂ fragments. Antibody fragments and derivatives produced by genetic engineering techniques are also provided.

In one embodiment, the antibodies are specific for the polypeptides of the present invention and do not cross-react with other proteins. Screening procedures by which such antibodies may be identified are well known, and may involve immunoaffinity chromatography, for example.

The antibodies of the invention can be used in assays to detect the presence of the polypeptides or fragments of the invention, either in vitro or in vivo. The antibodies also may be employed in purifying polypeptides or fragments of the invention by immunoaffinity chromatography.

Those antibodies that additionally can block binding of the polypeptides of the invention to the binding partner may be used to inhibit a biological activity that results from such binding. Such blocking antibodies may be identified using any suitable assay procedure, such as by testing antibodies for the ability to inhibit binding of FIL-1 theta to certain cells expressing the FIL-1 theta receptors. Alternatively, blocking antibodies may be identified in assays for the ability to inhibit a biological effect that results from polypeptides of the invention binding to their binding partners to target cells. Antibodies may be assayed for the ability to inhibit FIL-1 theta-mediated or binding partner-mediated cell lysis, for example.

Such an antibody may be employed in an in vitro procedure, or administered in vivo to inhibit a biological activity mediated by the entity that generated the antibody. Disorders caused or exacerbated (directly or indirectly) by the interaction of the polypeptides of the invention with the binding partner thus may be treated. A therapeutic method involves in vivo administration of a blocking antibody to a mammal in an amount effective in inhibiting a binding partner-mediated biological activity. Monoclonal antibodies are generally preferred for use in such therapeutic methods. In one embodiment, an antigen-binding antibody fragment is employed.

Antibodies may be screened for agonistic (i.e., ligand-mimicking) properties. Such antibodies, upon binding to cell surface receptor, induce biological effects (e.g., transduction of biological signals) similar to the biological effects induced when IL-1 binds to cell surface IL-1 receptors. Agonistic antibodies may be used to activate vascular endothelial cells and lymphocytes, induce local tissue destruction and fever (Janeway et al., 1996), stimulate macrophages and vascular endothelial cells to produce IL-6, and upregulate molecules on the surface of vascular endothelial cells.

Compositions comprising an antibody that is directed against polypeptides of the invention, and a physiologically acceptable diluent, excipient, or carrier, are provided herein. Suitable components of such compositions are as described above for compositions containing polypeptides of the invention.

Also provided herein are conjugates comprising a detectable (e.g., diagnostic) or therapeutic agent, attached to the antibody. Examples of such agents are presented above. The conjugates find use in in vitro or in vivo procedures.

The following examples are provided to further illustrate particular embodiments of the invention, and are not to be construed as limiting the scope of the present invention.

### EXAMPLE 1

### Isolation of the FIL-1 Theta and Identification of Distribution

Two exons of a gene with apparent homology to the IL-1 family were discovered in a genomic database. PCR primers were designed based upon the sequence of the two exons. The two exons correspond to exons 5 and 6 (the last two exons) of a typical IL-1 family member. The designed PCR amplification primers had the following sequences:

Forward Primer: AGA AGA TCT GCA TAC TTC CTA (from exon 5) SEQ ID NO:5

Reverse Primer: TGA GCA GGA TGA GCT TGG T (from exon 6) SEQ ID NO:6

The primers were synthesized and used to PCR amplify a panel of Clontech Human Tissue cDNA and Biochain Human Adult Normal Skin cDNA. A 396 bp fragment was the expected PCR product. This is based upon the size of IL-1 family members and the size of a correctly spliced version of exon 5 and exon 6. The PCR procedures involved thirty PCR amplification cycles with a 60° C. annealing temperature and included using 25 picomoles of primers per reaction and Hotstart Taq polymerase (Qiagen, Valencia, Calif.). A PCR product of predicted size was obtained from skin and tonsil first-strand cDNA and the following Clontech cDNA sources were negative: small intestine, liver, brain, lung fetal liver, lymph, bone marrow, kidney, pancreas, skeletal muscle, heart, prostate, ovary, thymus, spleen, leukocytes, testis, placenta, and colon. Following the thirty cycle PCR procedure, the same panel was screened again using a forty cycle PCR procedure. The forty cycle PCR amplification procedure resulted in the positive identification of lung and placenta. Human small airway epithelial cDNA from unstimulated cells and cells stimulated under various conditions were detected as positive after forty PCR amplification cycles under the same conditions. The sequence of the Biochain Human Adult Normal skin PCR product matched the sequence predicted for a correctly spliced product of exon 5 and exon 6. The sequence was determined by standard double stranded sequencing of the composite sequence and of additional sequences obtained from PCR and 5′ RACE reactions.

Exon 6 contains the translational stop codon. In order to extend the cDNA clone in the 5′ direction to identify the mature N-terminus, 5′RACE was performed on Clontech Marathon Ready Lung cDNA using the following primers:

Vector entry forward primer: CCA TCC TAA TAC GAC TCA CTA TAG GGC (SEQ ID NO:7)

Reverse Gene specific Primer: CTC CAG CTG TAG GGA AGG (SEQ ID NO:8) (or SEQ ID NO:6)

A nested reaction was then performed using the first round reaction product as a template with a nested vector entry primer as follows:

Forward Primer: ACT CAC TAT AGG GCT CGA GCG GC (SEQ ID NO:9)

Reverse gene specific prime: TCT TCT GTC TCC ACA CAT GC, (SEQ ID NO:10); or,

CAA GGC TAA AAC GCA GTT TC (SEQ ID NO:11)

The entire nested PCR reactions were subcloned into Novagen pT7Blue3 and transformants were screened to identify potential positives. Miniprep DNA from twenty-two transformants was sequenced. Clone Lung47 resembled the correctly spliced skin1cDNA and further extended the sequence 171 bp upstream before reaching a stop codon. The transformant contains a correctly spliced “exon 4”, but the transformant is incorrectly spliced upstream of this.

In further testing, a custom pCMVSport cDNA library made by Life Technologies from Immunex small airway epithelial RNA was screened for the presence of exon 5 and exon 6 by PCR amplifying with the primers of SEQ ID NO:5 and SEQ ID NO:6. This library source was positive so 5′ RACE was performed. In the first round the following primers were used:

Vector entry forward primer: CAG GAA ACA GCT ATG ACC AT (SEQ ID NO:12)

Reverse gene specific primer: SEQ ID NO:6 or SEQ ID NO:8. A nested reaction was then performed using the first round reaction product as a template with the following primers:

Vector entry forward primer: CTA TTT AGG TGA CAC TAT AGA A (SEQ ID NO:13)

Reverse gene specific primer SEQ ID NO:10 or SEQ ID NO:11

The products of the entire nested PCR reactions were subcloned into Novagen pT7Blue3 and transformants were screened to identify potential positives. Miniprep DNA from twelve transformants was sequenced. All twelve clones, including Clone SAE48, were identical. They were homologous to skin1cDNA and extended this sequence 139 bp upstream and included the exon 4 identified in clone Lung47 as well as an “exon 3” with an in-frame initiating methionine.

5′ RACE was repeated on the SAE library in a manner similar to the RACE reaction above with the exception that the reverse primers were located closer to the N-terminus. Nine clones were sequenced and they were identical to SAE48.

Full Length FIL-1 Theta was amplified from Clontech Placenta cDNA with primers designed from SAE48.

Sequencing data further established that FIL-1 Theta exhibits polymorphisms at amino acid 44, which is threonine or isoleucine, and at amino acid 51, which is aspartic acid or alanine, where the initiating methionine is designated +1. As indicated below, there are three alleles at the codon for amino acid 44. The corresponding nucleotides encoding the polymorphisms are as follows:

For amino acid 44: Threonine (nucleotide 130 to 132)-ACA; Isoleucine (also nucleotide 130 to 132) ATA or ATC

For amino acid 51: Aspartic Acid (nucleotide 151 to 153) GAC; Alanine (also nucleotide 151 to 153)-GCC.

The polymorphisms were detected in placenta sources, lung sources and SAE with the following frequency:

Isoleucine at amino acid 44 encoded by ATC was detected in one placenta cDNA clone.

Isoleucine at amino acid 44 encoded by ATA was detected in four samples including two lung, Lung47 and Lung19 (genomic), placenta cDNA and ac016724 (genomic).

Threonine at amino acid 44 encoded by ACA was detected in 6 placenta cDNA samples and small airway epithelial cDNA.

### EXAMPLE 2

### Use of Purified FIL-1 Theta in Polypeptide Specific ELISA

Serial dilutions of FIL-1 theta (in 50 mM NaHCO₃, brought to pH 9 with NaOH) are coated onto Linbro/Titertek 96 well flat bottom E.I.A. microtitration plates (ICN Biomedicals Inc., Aurora, Ohio) at 100:1/well. After incubation at 4° C. for 16 hours, the wells are washed six times with 200:1 PBS containing 0.05% Tween-20 (PBS-Tween). The wells are then incubated with FLAG-binding partner at 1 mg/ml in PBS-Tween with 5% fetal calf serum (FCS) for 90 minutes (100:1 per well), followed by washing as above. Next, each well is incubated with the anti-FLAG (monoclonal antibody M2 at 1 mg/ml in PBS-Tween containing 5% FCS for 90 minutes (100:1 per well), followed by washing as above. Subsequently, wells are incubated with a polyclonal goat anti-mIgG1-specific horseradish peroxidase-conjugated antibody (a 1:5000 dilution of the commercial stock in PBS-Tween containing 5% FCS) for 90 minutes (100:1 per well). The HRP-conjugated antibody is obtained from Southern Biotechnology Associates, Inc., Birmingham, Ala. Wells then are washed six times, as above.

For development of the ELISA, a substrate mix [100:1 per well of a 1:1 premix of the TMB Peroxidase Substrate and Peroxidase Solution B (Kirkegaard Perry Laboratories, Gaithersburg, Md.)] is added to the wells. After sufficient color reaction, the enzymatic reaction is terminated by addition of 2 N H₂SO₄ (50:1 per well). Color intensity (indicating ligand receptor binding) is determined by measuring extinction at 450 nm on a V Max plate reader (Molecular Devices, Sunnyvale, Calif.).

### EXAMPLE 3

### Monoclonal Antibodies that Bind Polypeptides of the Invention

This example illustrates a method for preparing monoclonal antibodies that bind FIL-1 theta polypeptide. Suitable immunogens that may be employed in generating such antibodies include, but are not limited to, purified FIL-1 theta polypeptide (e.g. SEQ ID NO:2 or SEQ ID NO:4), or an immunogenic fragment or variant thereof such as the extracellular domain, or fusion proteins containing FIL-1 theta (e.g., a soluble FIL-1 theta/Fc fusion protein).

Purified FIL-1 theta polypeptide can be used to generate monoclonal antibodies immunoreactive therewith, using conventional techniques such as those described in U.S. Pat. No. 4,411,993. Briefly, mice are immunized with FIL-1 theta immunogen emulsified in complete Freund's adjuvant, and injected in amounts ranging from 10-100 μg subcutaneously or intraperitoneally. Ten to twelve days later, the immunized animals are boosted with additional FIL-1 theta emulsified in incomplete Freund's adjuvant. Mice are periodically boosted thereafter on a weekly to bi-weekly immunization schedule. Serum samples are periodically taken by retro-orbital bleeding or tail-tip excision to test for FIL-1 theta antibodies by dot blot assay, ELISA (Enzyme-Linked Immunosorbent Assay) or inhibition of FIL-1 theta receptor binding.

Following detection of an appropriate antibody titer, positive animals are provided one last intravenous injection of FIL-1 theta in saline. Three to four days later, the animals are sacrificed, spleen cells harvested, and spleen cells are fused to a murine myeloma cell line, e.g., NS1 or preferably P3x63Ag8.653 (ATCC CRL 1580). Fusions generate hybridoma cells, which are plated in multiple microtiter plates in a HAT (hypoxanthine, aminopterin and thymidine) selective medium to inhibit proliferation of non-fused cells, myeloma hybrids, and spleen cell hybrids.

The hybridoma cells are screened by ELISA for reactivity against purified FIL-1 theta by adaptations of the techniques disclosed in Engvall et al., Immunochem., 8:871, 1971 and in U.S. Pat. No. 4,703,004. A preferred screening technique is the antibody capture technique described in Beckmann et al., J. Immunol., 144:4212, 1990. Positive hybridoma cells can be injected intraperitoneally into syngeneic BALB/c mice to produce ascites containing high concentrations of anti-FIL-1 theta monoclonal antibodies. Alternatively, hybridoma cells can be grown in vitro in flasks or roller bottles by various techniques. Monoclonal antibodies produced in mouse ascites can be purified by ammonium sulfate precipitation, followed by gel exclusion chromatography. Alternatively, affinity chromatography based upon binding of antibody to Protein A or Protein G can also be used, as can affinity chromatography based upon binding to FIL-1 theta.

### EXAMPLE 4

### Tissue Distribution of FIL-1 Theta mRNA

The tissue distribution of FIL-1 theta mRNA was investigated using first strand cDNAs from multiple human tissue sources (Clontech, Palo Alto, Calif.) withFIL-1 theta specific primers. The PCR reactions generated PCR products having the predicted size. FIL-1 theta was detected in tonsil, skin, lung, placenta, and small airway epithelium.

### EXAMPLE 5

### Binding Assay for FIL-1 Theta

Full length FIL-1 theta can be expressed and tested for the ability to bind FIL-1 theta receptors. The binding assay can be conducted as follows.

A fusion protein comprising a leucine zipper peptide fused to the N-terminus of a soluble FIL-1 theta polypeptide (LZ-FIL-1 theta) is employed in the assay. An expression construct is prepared, essentially as described for preparation of the FLAG⁷(FIL-1 theta) expression construct in Wiley et al., Immunity, 3:673-682, 1995, hereby incorporated by reference, except that DNA encoding the FLAG⁷ peptide was replaced with a sequence encoding a modified leucine zipper that allows for trimerization. The construct, in expression vector pDC409, encodes a leader sequence derived from human cytomegalovirus, followed by the leucine zipper moiety fused to the N-terminus of a soluble FIL-1 theta polypeptide. The LZ-FIL-1 theta is expressed in CHO cells, and purified from the culture supernatant.

The expression vector designated pDC409 is a mammalian expression vector derived from the pDC406 vector described in McMahan et al. EMBO J., 10:2821-2832, 1991, hereby incorporated by reference. Features added to pDC409 (compared to pDC406) include additional unique restriction sites in the multiple cloning site (mcs); three stop codons (one in each reading frame) positioned downstream of the mcs; and a T7 polymerase promoter, downstream of the mcs, that facilitates sequencing of DNA inserted into the mcs.

For expression of full length human FIL-1 theta protein, the entire coding region (i.e., the DNA sequence presented in SEQ ID NO:3) is amplified by polymerase chain reaction (PCR). The template employed in the PCR is the cDNA clone isolated from a (pancreatic tumor) cDNA library, as described in example 1. The isolated and amplified DNA is inserted into the expression vector pDC409, to yield a construct designated pDC409-FIL-1 theta.

LZ-FIL-1 theta polypeptide is employed to test the ability to bind to host cells expressing recombinant or endogenous FIL-1 theta receptors, as discussed above. Cells expressing FIL-1 theta receptor are cultured in DMEM supplemented with 10% fetal bovine serum, penicillin, streptomycin, and glutamine. Cells are incubated with LZ-FIL-1 theta (5 mg/ml) for about 1 hour. Following incubation, the cells are washed to remove unbound LZ-FIL-1 theta and incubated with a biotinylated anti-LZ monoclonal antibody (5 mg/ml), and phycoerythrin-conjugated streptavidin (1:400), before analysis by fluorescence-activated cell scanning (FACS). The cytometric analysis is conducted on a FACscan (Beckton Dickinson, San Jose, Calif.).

The cells expressing FIL-1 theta receptors will show significantly enhanced binding of FIL-1 theta, compared to the control cells not expressing FIL-1 theta receptors.

### EXAMPLE 6

### Isolating Mouse FIL-1 Theta

This example describes the identification and isolation of mouse FIL-1. A database of translated mouse genomic DNA sequences was searched (tblastn) using the entire human FIL-1 eta amino acid sequence (See WO 00/71720). Among the sequences identified as relevant were those that encode putative exons which most closely resembled exons 5 and 6 of human FIL-1 theta. Primers that annealed to these exons were prepared.

The following primers were used to screen mouse first-strand cDNA's using PCR procedures:

GTC TGT ATC CTT CCT AAC CGA GGC CTA GAC (SEQ ID NO:16) is a 5′ sense primer situated at the 5′ end of exon5. GTA TGG GTG GAG GGT TCA CTC TCT TTG GTG (SEQ ID NO:17) is a 3′ antisense primer situated near the 3′ end of exon6.

The cDNA sources that were screened included macrophage cell line RAW, LPS-stimulated RAW, testis, liver, lung, skeletal muscle, day 17 embryo, kidney, and LPS-stimulated RAW cell lambda phage library. Reaction conditions were as follows: Reaction volume of 50 μL including 1.0 ng cDNA or 1.0 μL lambda phage library, 12.5 pmol of each primer, 200 μM each dNTP, and 1.25 Units HotStarTaq (Qiagen Cat#203205). The PCR temperature cycling was as follows: 1 cycle at 95° C., for 15 min., 50° C. for 45 sec., and 72° C. for 1 min; 33 cycles at 95° C. for 45 sec., 50° C., for 45 sec., and 72° C., for 1 min.; and, 1 cycle at 95° C. for 45 sec., 50° C. for 45 sec., and 72° C., 5 min.

After the PCR amplification step, 10 μL each PCR reaction product was analyzed on a 1.5% TAE agarose gel and the products visualized under UV light following staining with ethidium bromide. A 308 bp product, corresponding to correct splicing of exons 5 and 6, was observed for lung, skeletal muscle, day 17 embryo, and kidney first-strand cDNA's. The results validated the assumption that these putative exons are indeed expressed as a spliced RNA transcript. The above described primers amplify a 573 bp target for genomic DNA.

In order to amplify exons 3 and 4 using 5′ RACE PCR Amplification, nested PCR primers (3′ antisense) were prepared. The outer primer was SEQ ID NO:17 and the nested primer was as follows:

TGT CTT TAC ACA CGC CAG GCA GCA ACT TCC-(SEQ ID NO:18), which lies at the end of exon 5. 5′ RACE was on adaptored cDNA from day 17 embryo (Marathon-ready cDNA, day 17 embryo; Clontech Cat #7460-1). Two nested rounds of PCR were performed.

For 5′ RACE-Round 1, primer of SEQ ID NO:17 and anchored primer of SEQ ID NO:7 were used. The 5′ RACE Round 1 reaction conditions were as follows: A volume of 50 μL including 0.5 ng adaptored cDNA, 12.5 pmol of each primer, 200 μM of each dNTP, and 1.25 Units HotStarTaq (Qiagen Cat#203205). The temperature cycling was as follows:

1 cycle at 95° C. for 15 min., 50° C. for 45 sec., and 72° C. for 1 min. Then 33 cycle at 95° C. for 45 sec., 50° C. for 45 sec., and 72° C. for 1 min.; and, 1 cycle at 95° C. for 45 sec., 50 ° C. for 45 sec., 50° C. for 45 sec., and 72° C. for 5 min. 5′ RACE Round 2 reaction conditions were as follows:

The primer of SEQ ID NO:18 and anchored primer of SEQ ID NO:9 were used. The Round 2 reaction was carried out by diluting the product of Round 1 by 1:10 in TE and preparing a mixture of 50 μL including 0.5 ng adaptored cDNA, 12.5 pmol of each primer, 200 μM of each dNTP, and 1.25 Units HotStarTaq (Qiagen Cat#203205). The temperature cycling was as follows: 1 cycle at 95° C. for 15 min., 60° C. for 45 sec., and, 72° C. for 1 min. Then 33 cycles at 95° C. for 45 sec., 60° C. for 45 sec., and 72° C. for 1 min. Then 1 cycle at 95° C. 45 sec., 60° C. for 45 sec., and 72° C. for 5 min.

Following the amplification, 10 μl of the products from Round 1 and Round 2 were analyzed on a 0.9% TAE gel. A band of about 300 bp was observed from the second round of RACE. The entire second round RACE reaction was subcloned into pT7Blue-3 and the sequences of 5 clones were analyzed. Three clones contained DNA which encodes an ORF homologous to exons 3 and 4 of human FIL-1 theta, indicating that the remainder of mouse FIL-1 theta sequence had been obtained.

A mouse full-length FIL-1 theta clone was amplified by preparing a primer pair that spans the entire predicted mouse FIL-1 theta and using them to amplify adaptored day 17 embryo cDNA. The 5′ sense primer was GCA TTA GAA TCC AGG CAC CAG GAA CTA CAG (SEQ ID NO:19) and lies 30 nt 5′ of the putative ATG. The 3′ antisense primer was TCC TTA TTC TGC TTT CCA GAG ATG CTG AGC (SEQ ID NO:20). The reaction conditions were as follows:

The PCR sample included 0.5 ng adaptored cDNA, 12.5 pmol of each primer 200 μM of each dNTP, and 0.5 μL of 16:1 KlenTaq:Vent. The temperature cycling was as follows: 1 cycle at 98° C. for 3 min., 55° C. at 45 sec. for 72° C. for 1 min. Then 28 cycles at 98° C. for 45 sec., 55° C. for 45 sec., and 72° C. for 45 sec. Then 1 cycle at 98° C. for 45 sec., 55° C. for 45 sec., and 72° C. for 5 min.

Following the amplification reaction, 5 μL of the reaction product was analyzed on a 1.0% TAE agarose gel. A 598 bp band was observed, which corresponds to the predicted size. This product was subcloned into pT7Blue-3 and 4 clones were sequenced. The sequence of each was identical and what was predicted for mouse FIL-1 theta.

What is claimed is:

1. An isolated DNA selected from the group consisting of:
(a) DNA comprising SEQ ID NO:1;
(b) DNA comprising SEQ ID NO:3, with the proviso that nucleotides 130-132 are selected from the group consisting of ACA, ATA and ATC;
(c) DNA comprising SEQ ID NO:3, with the proviso that nucleotides 151-153 are selected from the group consisting of GAC and GCC.
(d) DNA comprising SEQ ID NO:3;
(e) DNA comprising nucleotides 29-487 SEQ ID NO:14
(f) DNA that encodes a polypeptide comprising the amino acid sequence of SEQ ID NO:2;
(g) DNA that encodes a polypeptide comprising the amino acid sequence of SEQ ID NO:4;
(h) DNA that encodes a polypeptide comprising the amino acid sequence of SEQ ID NO:4, with the proviso that the amino acid at residue 44 is selected from the group consisting of threonine and isoleucine and the amino acid at residue 51 is selected from the group consisting of aspartic acid and alanine.
(i) DNA that encodes a polypeptide comprising the amino acid sequence of SEQ ID NO:15;
(j) DNA comprising a DNA that is degenerate from the DNA of (a) through (i)
(k) DNA comprising a DNA that is at least 80% identical to the DNA of (a)-(j); and,
(l) DNA comprising a DNA that encodes amino acids 1 through 152 of SEQ ID NO:4.

2. An isolated DNA comprising a DNA that is at least 85% identical to DNA that encodes the polypeptide of SEQ ID NO:4.

3. An isolated nucleic acid molecule comprising a DNA the encodes the polypeptide of SEQ ID NO:4.

4. An isolated nucleic acid molecule comprising a DNA the encodes the polypeptide of SEQ ID NO:4, except that amino acid at residue 44 is selected from the group consisting of threonine and isoleucine.

5. An isolated nucleic acid molecule comprising a DNA the encodes the polypeptide of SEQ ID NO:4, except that amino acid 51 is selected from the group consisting of aspartic acid and alanine.

6. An isolated nucleic acid molecule comprising a DNA the encodes the polypeptide of SEQ ID NO:4, except that amino acid at residue 44 is selected from the group consisting of threonine and isoleucine and amino acid 51 is selected from the group consisting of aspartic acid and alanine.

7. An isolated DNA comprising a fragment of a DNA that encodes the polypeptide of claim 3, wherein the fragment has IL-1 activity.

8. An isolated DNA comprising a nucleic acid that encodes a polypeptide that is at least 85% identical to the polypeptide of SEQ ID NO:15.

9. An isolated DNA comprising a nucleic acid that encodes the polypeptide of SEQ ID NO:15.

10. An isolated DNA comprising a nucleic acid that encodes a fragment of the polypeptide of claim 6, wherein the fragment has IL-1 activity.

11. A vector comprising the DNA of claim 3.

12. A vector comprising the DNA of claim 6.

13. A vector comprising the DNA of claim 10.

14. A host cell comprising a vector of claim 12.

15. A host cell comprising a vector of claim 14.

16. A method for producing a polypeptide, the method comprising culturing a host cell of claim 14 under conditions promoting expression of the polypeptide.

17. A method for producing a polypeptide, the method comprising culturing a host cell of claim 15 under conditions promoting expression of the polypeptide.

18. An isolated polypeptide comprising a polypeptide encoded by any of the DNAs of claim 1.

19. An isolated polypeptide comprising the amino acid sequence of SEQ ID NO:4.

20. An isolated polypeptide comprising the amino acid sequence of SEQ ID NO:4, except that the amino acid at 44 is selected from the group consisting of threonine and isoleucine and the amino acid at 51 is selected from the group consisting of aspartic acid and alanine.

21. An isolated polypeptide comprising a polypeptide that is at least 80% identical to a polypeptide of claim 20.

22. An isolated polypeptide comprising the amino acid sequence of SEQ ID NO:15.

23. An isolated polypeptide that is a fragment of the polypeptide of SEQ ID NO:4, wherein the fragment binds an IL-1 receptor family member.

24. An isolated polypeptide that is a fragment of the polypeptide of claim 20 wherein the fragment binds an IL-1 receptor family member.

25. An antibody that binds to a polypeptide of claim 20.

26. An antibody that binds to a polypeptide of SEQ ID NO:15.

27. An isolated antibody according to claim 25, wherein the antibody is a monoclonal antibody.

28. An oligomer comprising a polypeptide of claim 20.

29. An oligomer comprising a polypeptide of claim 24.

30. A method for identifying compounds that alter FIL-1 theta activity, the method comprising:
(a) mixing a test compound with the polypeptide selected from the group consisting of:
(i) a polypeptide comprising the amino acid sequence of SEQ ID NO:4, except that the amino acid at 44 is selected from the group consisting of threonine and isoleucine and the amino acid at 51 is selected from the group consisting of aspartic acid and alanine;. and,
(ii) a polypeptide that is a fragment of the polypeptide of SEQ ID NO:4, wherein the fragment binds an IL-1 receptor family member.
(b) determining whether the test compound alters FIL-1 theta activity of the polypeptide.

31. A method for identifying compounds that inhibit the binding activity of FIL-1 theta polypeptides, the method comprising:
(a) mixing a test compound with a polypeptide selected from the group consisting of:
(i) a polypeptide comprising the amino acid sequence of SEQ ID NO:4, except that the amino acid at 44 is selected from the group consisting of threonine and isoleucine and the amino acid at 51 is selected from the group consisting of aspartic acid and alanine;. and,
(ii) a polypeptide that is a fragment of the polypeptide of SEQ ID NO:4, wherein the fragment binds an IL-1 receptor family member and a binding partner of the polypeptide; and
(b) determining whether the test compound inhibits the binding activity of said polypeptide.
